(19) United States
(12) Patent Application Publication
(10) Pub. No.: US 2024/0336679 A1
(43) Pub. Date: Oct. 10, 2024

(54) METHODS FOR THE TREATMENT OF CANCER

(71) Applicants: INSTITUT REGIONAL DU CANCER DE MONTPELLIER, Montpellier Cedex 5 (FR); INSTITUT NATIONAL DE LA SANTE ET DE LA RECHERCHE MEDICALE, Paris (FR); UNIVERSITE DE MONTPELLIER, Montpellier (FR)

(72) Inventors: Laetitia LINARES, St Jean de Cornies (FR); Nelly FIRMIN, Saint Geniès des Mourgues (FR); Gabrielle MANTEAUX, Bésayes (FR)

(21) Appl. No.: 18/293,824

(22) PCT Filed: Aug. 5, 2022
(86) PCT No.: PCT/EP2022/072123

§ 371 Date: Jan. 31, 2024

(30) Foreign Application Priority Data
Aug. 6, 2021 (EP) 21306099.9

(51) Int. Cl. C07K 16/24; A61K 31/145; A61K 31/381; A61K 31/55; A61P 35/00; G01N 33/574

(52) U.S. Cl. CPC C07K 16/248; A61K 31/145; A61K 31/381; A61K 31/55; A61P 35/00; G01N 33/57496

(57) ABSTRACT

The present disclosure relates to an Interleukin-6 (IL-6) signaling inhibitor selected from an IL-6 inhibitor, an IL-6 receptor inhibitor, an IL-6/IL-6 receptor complex inhibitor, a gp130 inhibitor and a STAT3 inhibitor or a pharmaceutical composition comprising such an IL-6 signaling inhibitor. for use in a method for treating and/or preventing cancer in a subject in need thereof. wherein the subject has been previously classified as being affected with a cancer exhibiting recruitment of MDM2 to chromatin. The inventors have observed that cancers exhibiting recruitment of MDM2 to chromatin have been shown to secrete IL-6 that in turn acts on myoblast cells to activate serine synthesis. Following these surprising and unexpected observations. the inventors set up methods of treatment that are able to induce cancer cells death in subjects that have been previously classified as being affected with a cancer exhibiting recruitment of MDM2 to chromatin.

### TECHNICAL FIELD

The present disclosure relates to the field of cancer treatment. In particular, to therapeutic tools for treating cancer.

### TECHNICAL BACKGROUND

Cancer refers to a group of diseases characterized by the development of abnormal cells that divide uncontrollably and can infiltrate and destroy normal body tissue. Cancer is the second-leading cause of death in the world. Numerous therapies have been developed to treat the various cancer diseases. However, sometimes, cancer cells can overcome the efficacy of the anticancer therapies. It is thus important to specifically investigate and target the mechanism of cancer and offer new and more effective therapies.

Several cancers are caused by a disruption of the mouse double minute 2 (MDM2) oncoprotein. MDM2 is recognized as an essential component of the tumor suppressor p53 pathway that is frequently overexpressed in several types of human cancers (Biderman et al., 2012, Wade et al., 2013). Recently, it has been demonstrated that MDM2 may be also recruited to chromatin, independently of p53, to regulate a transcriptional program implicated in amino acid metabolism and more particularly in that of serine and glycine (Riscal et al. Mol Cell. 2016 Jun. 16; 62(6):890-902). MDM2 operates independently of p53 to control serine/glycine metabolism and sustain cancer growth. Serine/glycine metabolism supports the growth of cancer cells by contributing to their anabolic demands as well as by regulating their redox state (Locasale JW. Nat Rev Cancer. 2013 August; 13(8):572-83) and nucleotides synthesis (Cissé et al., Sci Transl Med. 2020 Jun. 10; 12(547)). At this day, it has been mainly investigated cancer therapy by using inhibitors of MDM2 p53-dependant interaction to specifically interfere with MDM2 function as a negative regulator of p53. However, these treatments are not completely effective.

Cancers exhibiting recruitment of MDM2 to chromatin, such as liposarcoma (LPS), are complex types of cancer which remain highly important to treat or prevent. WO2019106126 relates to methods for the diagnosis of subjects suffering from liposarcoma exhibiting recruitment of MDM2 to chromatin or resistant to inhibitors of p53 and MDM2 interaction and methods for the treatment of liposarcoma. It has been observed that serine synthesis pathway and serine uptake sustain the growth of cancer cells (Cissé et al., Sci Transl Med. 2020 Jun. 10; 12(547)).

Therefore, there remains a need to set up new pharmaceutical compositions and methods for treating and/or preventing cancer exhibiting recruitment of MDM2 to chromatin, in particular liposarcoma.There is a need to provide new methods of treatment which display a much stronger ability to induce cancer cells death than known methods, toward cancer cells exhibiting recruitment of MDM2 to chromatin, in particular liposarcoma cancer cells.

There is a need to provide methods of determining whether a subject is affected with a cancer exhibiting a recruitment of MDM2 to chromatin.

The present invention has for purpose to satisfy all or part of those needs.

### SUMMARY

According to one embodiment, the present disclosure relates to an Interleukin-6 (IL-6) signaling inhibitor selected from an IL-6 inhibitor, an IL-6 receptor inhibitor, an IL-6/IL-6 receptor complex inhibitor, a gp130 inhibitor and a STAT3 inhibitor, for use in a method for treating and/or preventing cancer exhibiting recruitment of MDM2 to chromatin in a subject in need thereof.

The present disclosure relates to an Interleukin-6 (IL-6) signaling inhibitor selected from an IL-6 inhibitor, an IL-6 receptor inhibitor, an IL-6/IL-6 receptor complex inhibitor, a gp130 inhibitor and a STAT3 inhibitor, for use in a method for treating and/or preventing cancer in a subject in need thereof, wherein the subject has been previously classified as being affected with a cancer exhibiting recruitment of MDM2 to chromatin.

The present disclosure concerns the use of an Interleukin-6 (IL-6) signaling inhibitor selected from an IL-6 inhibitor, an IL-6 receptor inhibitor, an IL-6/IL-6 receptor complex inhibitor, a gp130 inhibitor and a STAT3 inhibitor for preparing a medicament for treating and/or preventing cancer exhibiting MDM2 recruitment to chromatin in a subject in need.

The present disclosure concerns the use of an Interleukin-6 (IL-6) signaling inhibitor selected from an IL-6 inhibitor, an IL-6 receptor inhibitor, an IL-6/IL-6 receptor complex inhibitor, a gp130 inhibitor and a STAT3 inhibitor for preparing a medicament for treating and/or preventing cancer in a subject in need thereof, wherein the subject has been previously classified as being affected with a cancer exhibiting recruitment of MDM2 to chromatin.

The present disclosure pertains to a method for treating and/or preventing cancer exhibiting recruitment of MDM2 to chromatin in a subject in need thereof, wherein the said method comprises a step of administering to the said subject an Interleukin-6 (IL-6) signaling inhibitor selected from an IL-6 inhibitor, an IL-6 receptor inhibitor, an IL-6/IL-6 receptor complex inhibitor, a gp130 inhibitor and a STAT3 inhibitor.

The present disclosure pertains to a method for treating and/or preventing cancer in a subject in need thereof, wherein the subject has been previously classified as being affected with a cancer exhibiting recruitment of MDM2 to chromatin and wherein the said method comprises a step of administering to the said subject an Interleukin-6 (IL-6) signaling inhibitor selected from an IL-6 inhibitor, an IL-6 receptor inhibitor, an IL-6/IL-6 receptor complex inhibitor, a gp130 inhibitor and a STAT3 inhibitor.

The present disclosure also relates to a method for treating and/or preventing cancer in a subject in need thereof, wherein the said method comprises the steps of:
a) determining eligibility of the said subject to being administered the said treatment and/or prevention by detecting recruitment of MDM2 to chromatin in a biological sample previously obtained from the said subject, and
b) administering to the said subject an Interleukin-6 (IL-6) signaling inhibitor selected from an IL-6 inhibitor, an IL-6 receptor inhibitor, an IL-6/IL-6 receptor complex inhibitor, a gp 130 inhibitor and a STAT3 inhibitor when recruitment of MDM2 to chromatin has been detected at step a).

The present disclosure pertains to a method for treating and/or preventing cancer in a subject in need thereof, wherein the said method comprises the steps of:
a) determining eligibility of the said subject to being administered the said treatment and/or prevention by detecting recruitment of MDM2 to chromatin in a biological sample previously obtained from the said subject, and
b) administering to the said subject an Interleukin-6 (IL-6) signaling inhibitor selected from an IL-6 inhibitor, an IL-6 receptor inhibitor, an IL-6/IL-6 receptor complex inhibitor, a gp130 inhibitor and a STAT3 inhibitor when the said subject has been classified as being affected with a cancer exhibiting recruitment of MDM2 to chromatin at step a).

As shown in the examples herein, cancers exhibiting recruitment of MDM2 to chromatin, for instance liposarcoma, have been shown to secrete IL-6 protein that in turn acts on myoblast cells to activate serine synthesis. It has also been shown that, subsequently, serine is used by cancer cells to sustain their growth. This fully new observation was made even when the cancer cells were cultured on a serine/glycine depleted medium. Following these surprising and unexpected observations, the inventors set up methods of treatment that are able to induce cancer cells death in subjects that have been previously classified as being affected with a cancer exhibiting recruitment of MDM2 to chromatin. Especially, methods of treatment of the present disclosure may, in some embodiments thereof, involve drastic serine depletion of cancer cells. It is further shown in the examples that the methods disclosed herein display an improved activity compared to the known methods for treating cancer exhibiting recruitment of MDM2 to chromatin, in particular liposarcoma.

In some other embodiments, cancer cells exhibiting recruitment of MDM2 to chromatin as disclosed herein are further deprived of serine and glycine, which encompasses the cancer subject being deprived of exogenous serine and glycine

In some embodiments, a cancer exhibiting recruitment of MDM2 to chromatin as disclosed herein may be selected from the group comprising; bone cancer, brain cancer, ovary cancer, breast cancer, lung cancer, colorectal cancer, osteosarcoma, skin cancer, malignant hemopathies, pancreatic cancer, prostate cancer and liposarcoma.

In some embodiments, a cancer exhibiting recruitment of MDM2 to chromatin as disclosed herein may be liposarcoma.

In some embodiments, an IL-6 inhibitor as disclosed herein may be an anti-IL-6 antibody, or an anti-sense oligonucleotide directed to IL-6.

In some embodiments, an IL-6 receptor inhibitor as disclosed herein may be an anti-IL-6 receptor antibody, or an anti-sense oligonucleotide directed to IL-6 receptor.

In some embodiments, an IL-6/IL-6 receptor complex inhibitor as disclosed herein may be an anti-IL-6/IL-6 receptor complex antibody, or an anti-sense oligonucleotide directed to IL-6/IL-6 receptor complex.

In some embodiments, a gp130 inhibitor as disclosed herein may be selected from an anti-gp130 antibody, bazedoxifene and an anti-sense oligonucleotide directed to gp130.

In some embodiments, a STAT3 inhibitor may be C188-9 or Stattic.

In some embodiments, an IL-6/IL-6 receptor complex inhibitor or a gp130 inhibitor as disclosed herein may encompass an antimorphic form of IL-6/IL-6 receptor complex inhibitor or gp130 inhibitor.

In some embodiments, an anti-IL-6 antibody as disclosed herein may be a monoclonal anti-IL-6 antibody.

In some embodiments, an anti-IL-6 antibody as disclosed herein may be selected from sirukumab, siltuximab, olokizumab, or clazakizumab.

In some embodiments, an anti-IL-6 receptor antibody may be selected from tocilizumab, sarilumab and TZLS-501.

In some embodiments, an anti-IL-6/IL-6 receptor complex antibody may be TZLS-501.

In some embodiments, the subject in need thereof as disclosed herein may be further treated with a MDM2 inhibitor.

According to another embodiment, the present disclosure also relates to a pharmaceutical composition comprising (i) an IL-6 signaling inhibitor selected from an IL-6 inhibitor, an IL-6 receptor inhibitor, an IL-6/IL-6 receptor complex, a gp 130 inhibitor and a STAT3 inhibitor, and (ii) a pharmaceutically acceptable carrier, for use in a method for treating and/or preventing cancer exhibiting recruitment of MDM2 to chromatin in a subject in need thereof.

According to another embodiment, the present disclosure also relates to a pharmaceutical composition comprising (i) an IL-6 signaling inhibitor selected from an IL-6 inhibitor, an IL-6 receptor inhibitor, an IL-6/IL-6 receptor complex, a gp 130 inhibitor and a STAT3 inhibitor, and (ii) a pharmaceutically acceptable carrier, for use in a method for treating and/or preventing cancer in a subject in need thereof, wherein the subject has been previously classified as being affected with a cancer exhibiting recruitment of MDM2 to chromatin.

According to another embodiment, the present disclosure also relates to a pharmaceutical composition comprising (i) an IL-6 signaling inhibitor selected from an IL-6 inhibitor, an IL-6 receptor inhibitor, an IL-6/IL-6 receptor complex, a gp 130 inhibitor and a STAT3 inhibitor, and (ii) a pharmaceutically acceptable carrier, for use in a method for treating and/or preventing cancer exhibiting recruitment of MDM2 to chromatin in a subject in need thereof, wherein the cancer cells exhibiting recruitment of MDM2 to chromatin are deprived of serine and glycine.

According to another embodiment, the present disclosure also relates to a pharmaceutical composition comprising (i) an IL-6 signaling inhibitor selected from an IL-6 inhibitor, an IL-6 receptor inhibitor, an IL-6/IL-6 receptor complex, a gp 130 inhibitor and a STAT3 inhibitor, and (ii) a pharmaceutically acceptable carrier, for use in a method for treating and/or preventing cancer in a subject in need thereof, wherein the subject has been previously classified as being affected with a cancer exhibiting recruitment of MDM2 to chromatin and wherein the cancer cells exhibiting recruitment of MDM2 to chromatin are deprived of serine and glycine.

According to another embodiment, the present disclosure also relates to a method of treating a subject having a cancer exhibiting recruitment of MDM2 to chromatin, comprising at least the steps of:
(a) depriving cancer cells of serine and glycine, and
(b) administering to the subject a therapeutically effective amount of an IL-6 signaling inhibitor selected from an IL-6 inhibitor, an IL-6 receptor inhibitor, an IL-6/IL-6 receptor complex inhibitor, a gp130 inhibitor and a STAT3 inhibitor.

According to another embodiment, the present disclosure also relates to an in vitro method of determining whether a subject is affected with a cancer exhibiting a recruitment of MDM2 to chromatin, wherein said subject is intended for a therapy which comprises an IL-6 signaling inhibitor, comprising:
determining whether MDM2 is localized in the cancer cells nucleus of a biological sample obtained from the subject,
wherein if MDM2 is localized in the cancer cell nucleus of the biological sample, it indicates that the subject is affected by a cancer exhibiting recruitment of MDM2 to chromatin.

In some embodiments, a subject may be a human.

### DESCRIPTION OF THE FIGURES

FIGS. 1 show that cancer cell lines exhibiting recruitment of MDM2 to chromatin secrete IL-6. FIG. 1A shows that human liposarcoma, breast cancer, melanoma and pancreatic cell lines exhibiting recruitment of MDM2 to chromatin secrete measurable IL-6 in their culture medium. IL-6 concentration in the supernatant of the different tested cancer cell lines was measured by an immunoassay (TMB Elisa kit from Peprotech). Abscissa: Tested cell lines (from left to right) CFPAC; MDAMB468; SKMEL5; IB115; IB111; JURKAT; MCF7; ZR751; H1299; LNCAP; HPAC; MIAPACA. Ordinate:

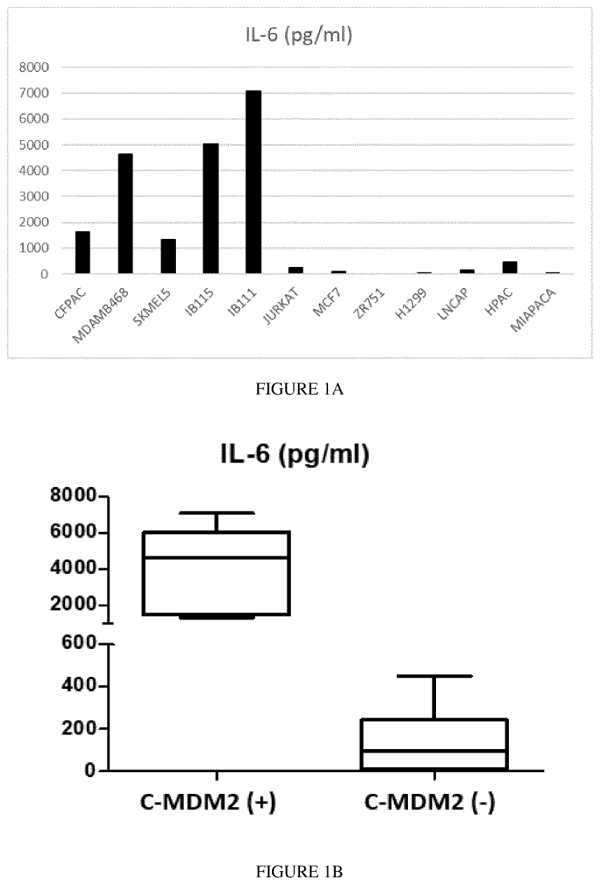

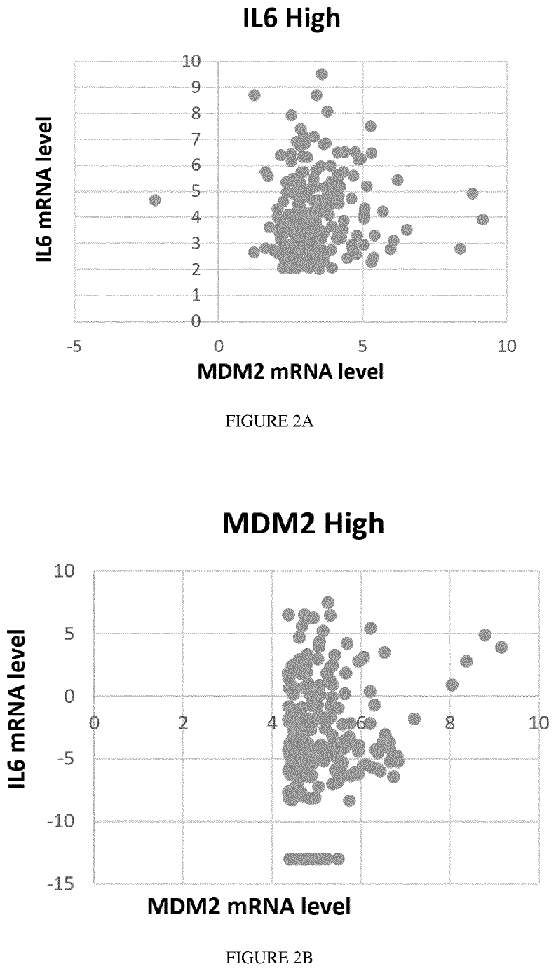

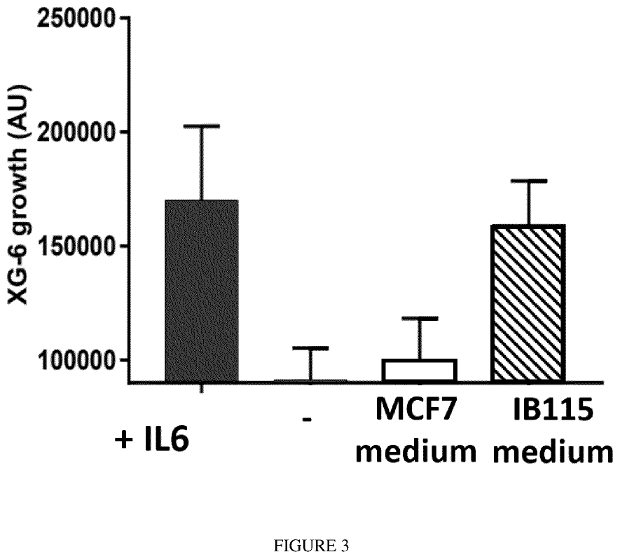

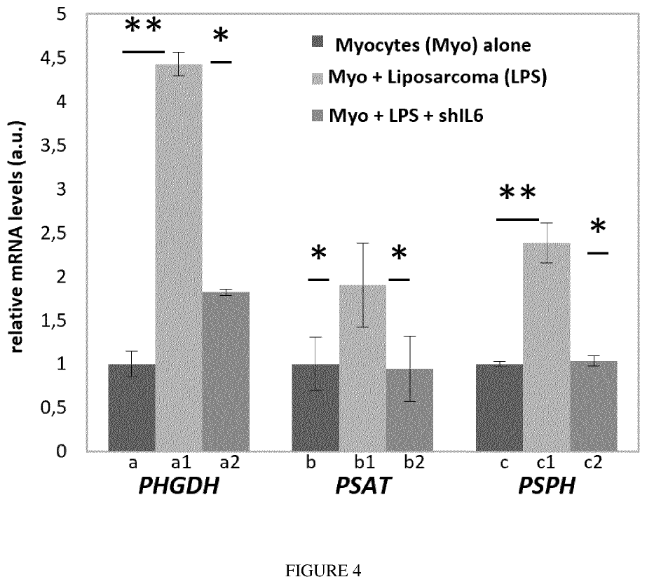

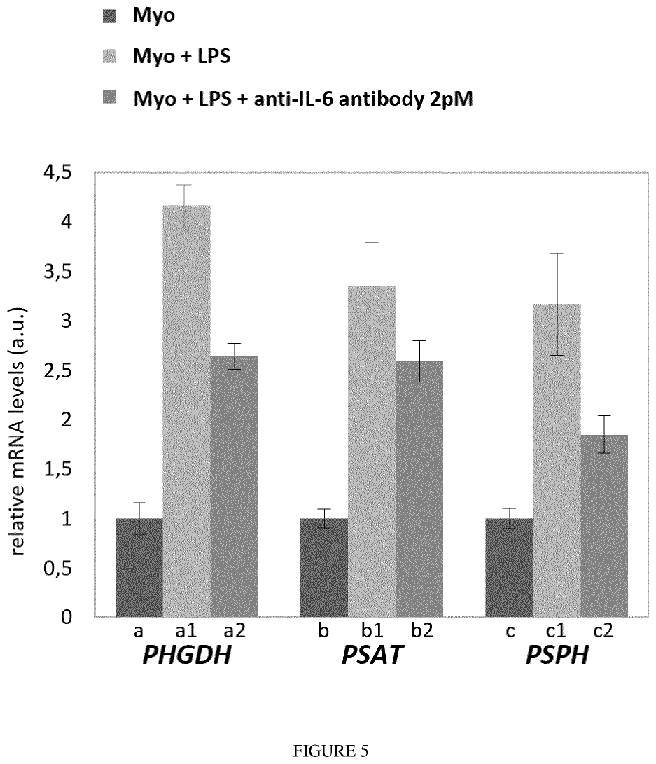

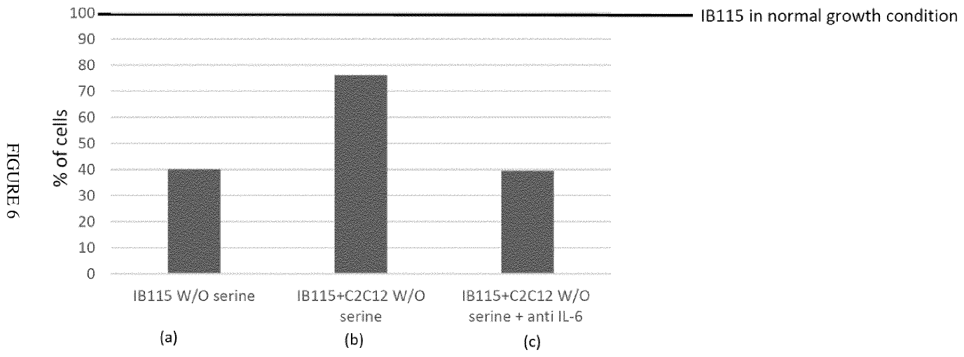

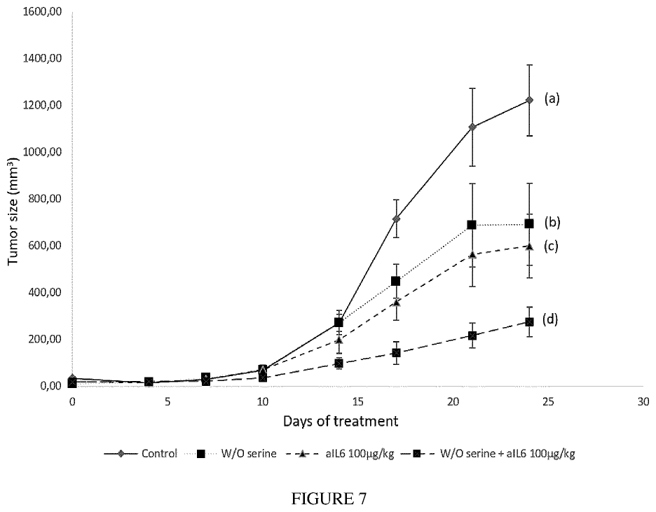

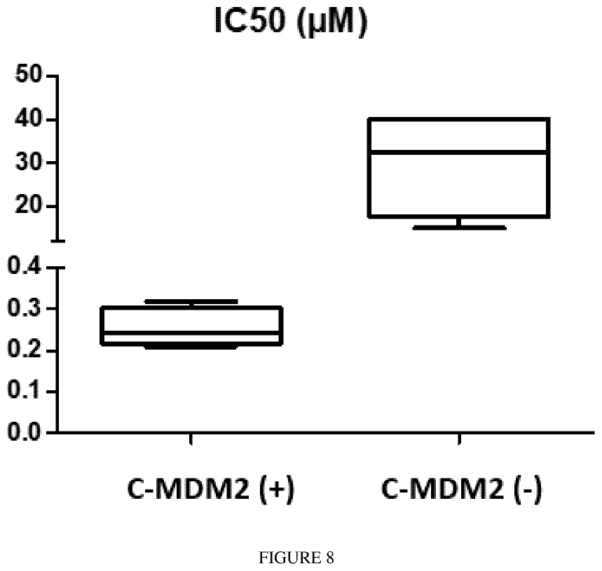

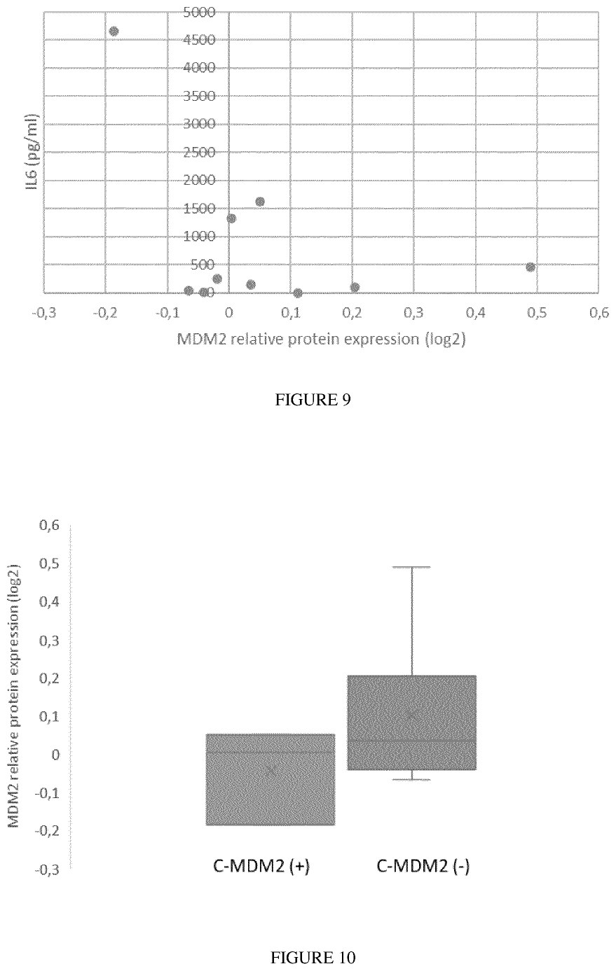

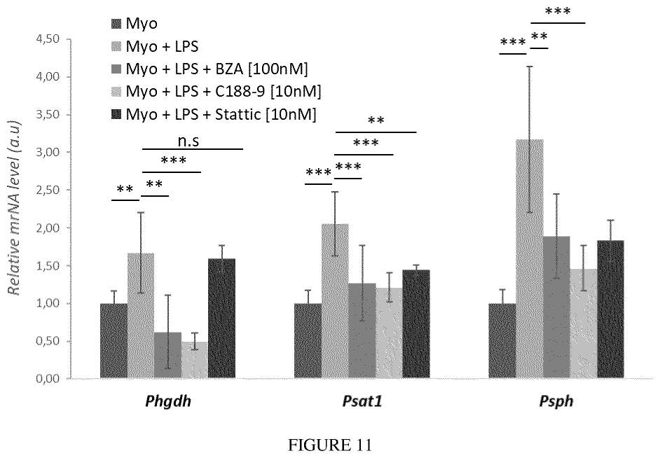

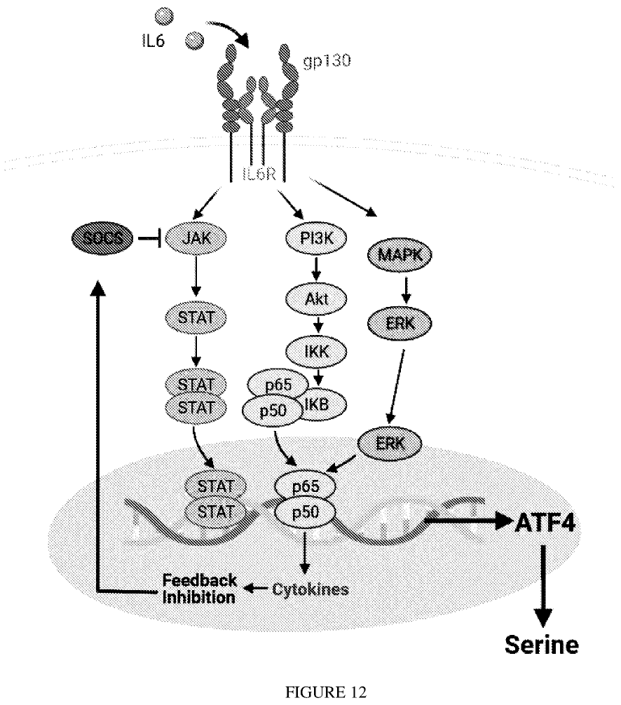

Concentration of IL-6 secreted by the tested cell lines in pg/ml. FIG. 1B shows that cancer cell lines exhibiting recruitment of MDM2 to chromatin (C-MDM2 (+)) secrete, on average, more than 4000 pg/ml of IL-6 whereas cancer cell lines without recruitment of MDM2 to chromatin (C-MDM2 (−)), secrete, on average, less than 100 pg/ml of IL-6. IL-6 expression is dependent on the localization of MDM2 in the cancer cell. Abscissa: (left to right): C-MDM2 (+) (Cancer cell lines exhibiting recruitment of MDM2 to chromatin); C-MDM2 (−) (Cancer cell lines without recruitment of MDM2 to chromatin). Ordinate: Concentration of IL-6 secreted by the tested cell lines in pg/ml.

FIGS. 2 show that IL-6 expression is independent on the level of MDM2 expression. FIG. 2A shows that cancer cell lines that express high level of IL-6, overexpress MDM2. Abscissa: IL-6 mRNA expression in cancer cell lines (arbitrary unit). Ordinate: MDM2 mRNA expression level in cancer cell lines. FIG. 2B shows that cancer cell lines that overexpress MDM2, do not necessarily express IL-6. Abscissa: IL-6 mRNA expression in cancer cell lines (arbitrary unit). Ordinate: MDM2 mRNA expression level in cancer cell lines.

FIG. 3 shows that the growth of IL-6 dependent cells is promoted by the IL-6 rich supernatant of liposarcoma cell lines. The IL-6 dependent cell line XG-6 was cultured in presence of recombinant IL-6 (control) or supernatant from two different cell lines (MCF7 breast cancer cells or IB115 liposarcoma cells) for 3 days. Abscissa (from left to right): XG-6 cell culture conditions: (a) XG-6 in RPMI medium (2 ml) in the presence of 2 ng/ml of recombinant IL-6 (positive control), (b) XG-6 in 2 ml RPMI medium only (negative control), (c) XG-6 in the supernatant RPMI medium (2 ml) from the preculture of MCF7 breast cancer cells, and (d) XG-6 in the supernatant RPMI medium (2 ml) from the preculture of IB 115 liposarcoma cells. Ordinate: Quantity of XG-6 cells (AU).

FIG. 4 shows that transcription of the genes involved in serine synthesis (PHGDH, PSAT, and PSPH) is enhanced by human IL-6 in mouse C2C12 myoblasts cocultured with human IB115 liposarcoma cells. Target genes analyzed by RT-qPCR in C2C12 myoblasts cultured alone or in coculture with liposarcoma IB115 cells transduced or not with shRNA to inactivate human IL-6 expression (shIL-6). All experiments are done with 50 000 C2C12 cells and 100 000 IB115 cells in 2 ml of DMEM medium. Abscissa: (from left to right) (a) PHGDH in myoblasts alone (Myo), (a1) PHGDH in myoblasts cocultured with IB115 liposarcoma cells (LPS), (a2) PHGDH in myoblasts cocultured with IB115 liposarcoma-shIL-6 cells, (b) PSAT in myoblasts alone, (b1) PSAT in myoblasts cocultured with IB115 liposarcoma cells, (b2) PSAT in myoblasts cocultured with IB115 liposarcoma-shIL-6 cells, and (c) PSPH in myocytes alone, (c1) PSPH in myocytes cocultured with IB115 liposarcoma cells, (c2) PSPH in myocytes cocultured with IB115 liposarcoma-shIL-6 cells. Ordinate: Relative quantity of mRNA levels (AU) of the target genes PHGDH, PSAT and PSPH. (mean+/SD; n=3 independent experiments). *p-value<0.005, **p-value<0.001 using non-parametric Mann-Whitney U tests.

FIG. 5 shows that in vitro treatment with an anti-IL-6 antibody decreases the IL-6 dependent enhancement of the serine synthesis way in mouse C2C12 myoblasts cocultured with human IB115 liposarcoma cells. All experiments are done with 50 000 C2C12 cells and 100 000 IB115 cells in 2 ml of DMEM medium. Target genes analyzed by RT-qPCR in C2C12 myoblasts cultured alone or in coculture with IB115 liposarcoma cells treated or not with anti-IL-6 antibody (2 ΣM). Abscissa: (from left to right) (a) PHGDH in myoblasts alone (Myo), (a1) PHGDH in myoblasts cocultured with IB115 liposarcoma cells (LPS), (a2) PHGDH in myoblasts cocultured with IB 115 liposarcoma cells and treated with anti-IL-6 antibody (2 ΣM), (b) PSAT in myoblasts alone, (b1) PSAT in myoblasts cocultured with IB115 liposarcoma cells, (b2) PSAT in myoblasts cocultured with IB115 liposarcoma cells and treated with anti-IL-6 antibody (2 μM), and (c) PSPH in myoblasts alone, (c1) PSPH in myoblasts cocultured with IB115 liposarcoma cells, (c2) PSPH in myoblasts cocultured with IB115 liposarcoma cells and treated with anti-IL-6 antibody (2 μM). Ordinate: Relative quantity of mRNA levels (AU) of the target genes PHGDH, PSAT and PSPH in each conditions (mean+/SD; n=3 independent experiments).

FIG. 6 shows that IL-6-stimulated C2C12 myoblasts provide liposarcoma cells with serine and sustain their growth in a serine-deprived medium. 60 000 IB115-GFP liposarcoma cells were cultured alone or in coculture with 20 000 RFP-C2C12 myoblast cells in 2 ml serine/glycine deprived DMEM medium with 5% serum. Cells were treated twice a week with anti-IL-6 antibody (1 μM) for 9 days. Abscissa (from left to right): (a) IB115 liposarcoma cells in a serine/glycine deprived DMEM medium with 5% serum, (b) IB115 liposarcoma cells cocultured with C2C12 myoblast cells in a serine/glycine deprived DMEM medium with 5% serum, (c) IB115 liposarcoma cells cocultured with C2C12 myoblast cells and treated with anti-IL-6 antibody (1 μM) in a serine/glycine deprived DMEM medium with 5% serum. Ordinate: Percentage of living cells in the culture at day 9.

FIG. 7 shows that a serine/glycine deprivation and a treatment with an anti-IL-6 antibody have a synergistic anti-tumor effect on a human liposarcoma tumor grafted on nude mice. Conditions of treatment of the mice: (a) Amino Acid diet (control) a mean value of 5 g/day, (b) Test diet (without serine/glycine) a mean value of 5 g/day, (c) Amino Acid diet (5 g/day) +intraperitoneal administration (i.p.) of a dose of 100 μg/kg of anti-IL-6 antibody, (d) Test diet (without serine/glycine) (5 g/day)+i.p injection of a dose of 100 μg/kg of anti-Il-6 antibody. Abscissa: Days of treatment of the mice. Ordinate: Tumor size in cubic millimeter (mm³) (n=10 independent experiments).

FIG. 8 shows that C-MDM2 degradation is more efficient in inducing cell death of cancer cells exhibiting a recruitment of MDM2 to chromatin (C-MDM2(+)) than cancer cells without recruitment of MDM2 to chromatin (C-MDM2(−)). Cancer cells exhibiting or not a recruitment of MDM2 to chromatin were treated for 72 hours with increasing concentrations of SP141. Ordinate: Mean of the measurement of IC₅₀ (μM) for SP141 on different cancer cell lines. Abscissa (left to right): C-MDM2(+) (Cancer cells exhibiting recruitment of MDM2 to chromatin—WSKMEL5, IB115, IB111); C-MDM2(−) (Cancer cells without recruitment of MDM2 to chromatin—MCDF7, ZR751, HPAC).

FIG. 9 shows that there is no correlation between the IL-6 secretion and the expression level of MDM2. Abscissa: MDM2 relative protein expression (log2) by cancer cell lines (https://sites.broadinstitute.org/ccle). Ordinate: Concentration of IL-6 secreted by the cancer cell lines in pg/ml.

FIG. 10 shows that there is no correlation between the localization and the level of expression of MDM2 in the cancer cell. MDM2 protein expression was compared between cell lines (https://sites.broadinstitute.org/ccle) with chromatin-bound MDM2 (C-MDM2 (+)) or cytoplasmic MDM2 (C-MDM2 (−)). Abscissa: (from left to right) C-MDM2 (+) (Cancer cell lines exhibiting recruitment of MDM2 to chromatin); C-MDM2 (−) (Cancer cell lines without recruitment of MDM2 to chromatin). Ordinate: Concentration of IL-6 secreted by the tested cell lines in pg/ml.

FIG. 11 shows that in vitro treatment with a STAT3 inhibtor (C188-9 or Stattic) or a gp130 inhibitor (Bazédoxifène) decreases the IL-6 dependent enhancement of the serine synthesis way in mouse C2C12 myoblasts cocultured with human IB115 liposarcoma cells. All experiments were done with 50 000 C2C12 cells and 100 000 IB115 cells in 2 ml of DMEM medium. Target genes analyzed by RT-qPCR in C2C12 myoblasts cultured alone or in coculture with IB 115 liposarcoma cells treated or not with Bazédoxifène (100 nM), C188-9 (10 nM) or Stattic (10 nM).

Abscissa for PHGDH (from left to right) (a) PHGDH in myoblasts alone (Myo), (a1) PHGDH in myoblasts cocultured with IB115 liposarcoma cells (Myo+LPS), (a2) PHGDH in myoblasts cocultured with IB115 liposarcoma cells treated with Bazédoxifène (100 nM), (a3) PHGDH in myoblasts cocultured with IB115 liposarcoma cells treated with C188-9 (10 nM), (a4) PHGDH in myoblasts cocultured with IB 115 liposarcoma cells treated with Stattic (10 nM).

Abscissa for PSAT1 (from left to right): (b) PSAT1 in myoblasts alone (Myo), (b1) PSAT1 in myoblasts cocultured with IB115 liposarcoma cells (Myo+LPS), (b2) PSAT1 in myoblasts cocultured with IB115 liposarcoma cells treated with Bazédoxifène (100 nM), (b3) PSAT1 in myoblasts cocultured with IB115 liposarcoma cells treated with C188-9 (10 nM), (b4) PSAT1 in myoblasts cocultured with IB115 liposarcoma cells treated with Stattic (10 nM).

Abscissa for PSPH (from left to right): (c) PSPH in myoblasts alone (Myo), (c1) PSPH in myoblasts cocultured with IB115 liposarcoma cells (Myo+LPS), (c2) PSPH in myoblasts cocultured with IB115 liposarcoma cells treated with Bazédoxifène (100 nM), (c3) PSPH in myoblasts cocultured with IB115 liposarcoma cells treated with C188-9 (10 nM), (c4) PSPH in myoblasts cocultured with IB115 liposarcoma cells treated with Stattic (10 nM).

Ordinate: Relative quantity of mRNA levels (AU) of the target genes PHGDH, PSAT and PSPH in each cited conditions (mean+/−SD; n=3 independent experiments).

FIG. 12 is an overview of the IL-6 pathway.

### DETAILED DESCRIPTION

### Definitions

The terms used in the present specification generally have their ordinary meaning in the art. Certain terms are discussed below, or elsewhere in the present disclosure, to provide additional guidance in describing the products and methods of the presently disclosed subject matter.

The following definitions apply in the context of the present disclosure.

As used in this specification and the appended claims, the singular forms “a”, “an” and “the” include plural referents unless the content clearly dictates otherwise.

As used herein, “administering” or “administered” means administration by any route, such as oral administration, administration as a suppository, topical contact, parenteral, intraperitoneal, intramuscular, intralesional, intrathecal, intranasal, subcutaneous or transmucosal (e.g., buccal, sublingual, palatal, gingival, nasal, vaginal, rectal, or transdermal) administration, or the implantation of a slow-release device, e.g., a mini-osmotic pump, to a subject, including parenteral. Parenteral administration includes intravenous, intramuscular, intra-arteriole, intradermal, subcutaneous, intraperitoneal, intraventricular, and intracranial.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 10% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, “antibody” refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. The term “antibody” includes both naturally occurring and non-naturally occurring antibodies. Specifically, “antibody” includes polyclonal and monoclonal antibodies, and monovalent and divalent fragments or portions thereof. Furthermore, “antibody” includes chimeric antibodies, wholly synthetic antibodies, single chain antibodies, and fragments or portions thereof. The antibody may be a human or nonhuman antibody. A nonhuman antibody may be humanized by recombinant methods to reduce its immunogenicity in human.

As used herein, the terms “antigen-binding portion”, “antibody portion” and “portion” of an antibody refer to one or more fragments of an antibody that retain the ability to specifically bind to an antigen (e.g., IL-6, IL-6R, STAT3 or gp130). It has been shown that the antigen-binding function of an antibody can be performed by fragments of a full-length antibody. Binding portions include Fab, Fab′, F (ab′)2, Fabc, Fv, single chains, and single-chain antibodies. Examples of binding portions encompassed within the term “antigen-binding portion” of an antibody include (i) a Fab fragment, a monovalent fragment consisting of the VL, VH, CL and CHI domains; (ii) a F (ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment which consists of a VH domain; and (vi) an isolated complementarity determining region (CDR).

As used herein the term “complementarity determining region” or “CDR” refers to the part of the two variable chains of antibodies (heavy and light chains) that recognize and bind to the particular antigen. The CDRs are the most variable portion of the variable chains and provide the antibody with its specificity. There are three CDRs on each of the variable heavy (VH) and variable light (VL) chains and thus there are a total of six CDRs per antibody molecule. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located.

As used herein, the term an “antibody heavy chain” refers to the larger of the two types of polypeptide chains present in all antibody molecules in their naturally occurring conformations.

As used herein, the term an “antibody light chain” refers to the smaller of the two types of polypeptide chains present in all antibody molecules in their naturally occurring conformations, κ and λ light chains refer to the two major antibody light chain isotypes.

As used herein, the term “blocking” antibody refers to an antibody suitable to prevent the interaction between a ligand and its receptor. Typically, a blocking antibody is directed against the said ligand, against the said receptor, or against the complex formed by the said ligand and the said receptor. The binding of the blocking antibody with an antigen selected among the said ligand, the said receptor, or the said ligand/receptor complex interferes with the binding of the said ligand to the said receptor, and especially prevents the binding of the said ligand to the said receptor. A “blocking” antibody according to the disclosure does not activated the said receptor for inducing a signal transduction. Specifically, a blocking anti-IL-6 signaling antibody refers to (i) antibodies that bind to IL-6 and block the interaction between IL-6 and its IL-6 receptor, (ii) antibodies that bind to IL-6 receptor and block the interaction between IL-6 and the IL-6 receptor, (iii) antibodies that bind IL-6/IL-6 receptor complex and block the interaction between the IL-6/IL-6 receptor complex and gp130, (iv) antibodies that bind to gp130 and block the interaction between IL-6, IL-6 receptor or IL-6/IL-6 receptor complex with gp130, or (v) antibodies that bind to STAT3 and block its phosphorylation.

It is understood that aspects and embodiments of the present disclosure described herein include “comprising”, “having”, and “consisting of,” aspects and embodiments. The words “have” and “comprise,” or variations such as “has,” “having,” “comprises,” or “comprising,” will be understood to imply the inclusion of the stated element(s) (such as a composition of matter or a method step) but not the exclusion of any other elements. The term “consisting of” implies the inclusion of the stated element(s), to the exclusion of any additional elements. The term “consisting essentially of” implies the inclusion of the stated elements, and possibly other element(s) where the other element(s) do not materially affect the characteristic(s) of the stated elements. It is understood that the different embodiments of the disclosure using the term “comprising” or equivalent cover the embodiments where this term is replaced with “consisting of” or “consisting essentially of”.

As used herein the term “days” consists of a space of time that elapses over a period of 24 hours, i.e., from 0:00 in the morning to 12:00 in the evening.

As used herein, the term “diet” refers to a food material containing amino acids with or without serine and glycine, carbohydrates and/or fats, which is used in the body of an organism to sustain growth, repair and vital processes and to furnish energy. Foods can also contain supplementary substances or additives, for example, minerals, vitamins and condiments (See Merriam-Webster's Collegiate Dictionary, 10th Edition, 1993).

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs, which are modified, by processes such as capping, polyadenylation, methylation, and editing, and proteins (e.g., IL-6, IL-6 receptor, STAT3 or gp130) modified by, for example, methylation, acetylation, phosphorylation, ubiquitination, SUMOylation, ADP-ribosylation, myristilation, and glycosylation.

An “expression inhibitor” refers to a natural or synthetic compound that has a biological effect to inhibit the expression of a gene.

As used herein, “framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues.

As used herein, the term “gene” refers to a nucleic acid (e.g., DNA or RNA) sequence that comprises partial length or entire length coding sequences necessary for the production of a polypeptide or precursor polypeptide, in particular for the production of IL-6, IL-R, STAT3 and gp130.

As used herein, the term “glycine” refers to an amino acid. An amino acid glycine may be used in the biosynthesis of proteins (CAS Registry Number® is 56-40-6). Glycine is not essential to the human diet, as it is biosynthesized in the body from the amino acid serine.

A “short hairpin RNA” or “shRNA” includes a short RNA sequence that makes a tight hairpin turn that can be used to silence gene expression via RNA interference. The shRNAs of the disclosure may be chemically synthesized or transcribed from a transcriptional cassette in a DNA plasmid. Non-limiting examples of shRNA include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In preferred embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides. Additional shRNA sequences include, but are not limited to, asymmetric shRNA precursor polynucleotides such as those described in PCT Publication Nos. WO 2006/074108 and WO 2009/076321. Suitable shRNA sequences can be identified, synthesized, and modified using any means known in the art for designing, synthesizing, and modifying siRNA sequences.

In certain embodiments, shRNAs may silence one or more IL-6 signaling pathway genes of interest, and preferably silence the expression of IL-6, IL-6R, STAT3 or gp 130. For instance, a sequence of a shRNA-IL-6 may be 5′-GACACTATTTTAATTATTTTTAA-3′.

A “small-interfering RNA” or “siRNA,” includes interfering RNA of about 15-60, 15-50, or 15-40 (duplex) nucleotides in length. Examples of siRNA include, without limitation, a double-stranded polynucleotide molecule assembled from two separate stranded molecules, wherein one strand is the sense strand and the other is the complementary antisense strand; a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule. As used herein, the term “siRNA” includes RNA-RNA duplexes as well as DNA-RNA hybrids (see, e.g., PCT Publication No. WO 2004/078941). siRNA may be chemically synthesized. siRNA can also be generated by cleavage of longer dsRNA (e.g., dsRNA greater than about 25 nucleotides in length) with the E. coli RNase III or Dicer. Preferably, dsRNA are at least 50 nucleotides to about 100, 200, 300, 400, or 500 nucleotides in length. The dsRNA can encode for an entire gene transcript or a partial gene transcript. In certain instances, siRNA may be encoded by a plasmid (e.g., transcribed as sequences that automatically fold into duplexes with hairpin loops).

The term “Interleukin-6 (IL-6) signaling inhibitor” refers to compounds or agents that selectively block or inactivate the IL-6 signaling pathway. As used herein, the term “selectively blocks or inactivates” refers to a compounds that preferentially bind to and block or inactivate IL-6, IL-6 receptor, IL-6/IL-6R complex, STAT3 and/or gp130. Especially compounds that block the interaction between IL-6 with its IL-6 receptor, IL-6 with gp130, IL-6 receptor with gp130, IL-6/IL-6 Receptor complex with gp130, or compounds that block the phosphorylation of STAT3 Typically, an IL-6 signaling inhibitor may encompass without limitation a polypeptide, an aptamer, an antibody or a portion thereof, an antisense oligonucleotide, i.e., a siRNA or a shRNA, or a ribozyme. Such types of inhibitor are further described in the present description.

The terms “IL-6/IL-6R complex” or “IL-6/IL-6 receptor complex” collectively refer to a complex formed by a soluble form of IL-6 and membrane-bound form IL-6 receptor alpha. In some embodiments of the present disclosure, “IL-6/IL-6R complex” refers to a complex formed by (i) a soluble form of IL-6 secreted by a cancer cell of a subject, and (ii) a membrane-bound form IL-6 receptor alpha of a myoblast cell of the same subject.

The term “MDM2” has its general meaning in the art and refers to mouse double minute 2 oncoprotein. The term “MDM2” also refers to E3 ubiquitin-protein ligase having the UniProtKB accession number Q00987.

The term “MDM2 inhibitor” refers to compounds that selectively block or inactivate the chromatin function of MDM2. As used herein, the term “selectively block or inactivate the chromatin function of MDM2” refers to a compound that preferentially binds to and block or inactivate the effect or function of MDM2 on chromatin with a greater affinity and potency, respectively, than other ubiquitin-protein ligases, or related enzymes or related transporters. Compounds that block or inactivate the chromatin function of MDM2 may also block or inactivate, enzymes related to PHGDH, PSAT, PSPH, or other members of the SLC1 family of proteins, as partial or full inhibitors, are contemplated. Typically, a MDM2 inhibitor is a small organic molecule, a polypeptide, an aptamer, an antibody, an intra-antibody, an oligonucleotide or a ribozyme. The MDM2 inhibitors are well-known in the art as illustrated by (Qin et ah, 2016; U.S. Pat. No. 8,329,723). MDM2 inhibitor may be selected from the group consisting of SP141, 6-Methoxy-1-naphthalen-2-yl-9H-P-carboline; SP141 nanoparticles (SP141NP); SP141-loaded IgG Fc-conjugated malcimidyl-poly (ethylene glycol)-co-poly (c-caprolactone) (Mal-PEG-PCL) nanoparticles (SP141FcNP); 6-Methoxy-1-quinolin-4-yl-9H-P-carboline; 6-Methoxy-1-naphthalen-1-yl-9H-P-carboline; 6-Methoxy-1-phenanthren-9-yl-9H-b-carboline; 7-Methoxy-1-phenanthren-9-yl-9H-P-carboline; miR-509-5p; shRNA and compounds described in Qin et ah, 2016; U.S. Pat. No. 8,329,723.

As used herein, “pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. By way of example of pharmaceutically acceptable carrier, sterile water, saccharides such as sucrose or saccharose, starches, sugar alcohols such as sorbitol, polymers such as PVP or PEG, lubricating agents, such as magnesium stearate, preservatives, dyeing agents or flavors can be mentioned.

The term “PHGDH” refers to phosphoglycerate dehydrogenase having the UniProtKB accession number 043175, Catalyzes the oxidation of 3-phosphoglycerate (3PG) to 3-phosphohydroxypyruvate (3P-Pyr), the first step of the serine biosynthesis pathway.

As used herein, the terms “prevent” or “preventing” with respect to a disease or disorder relate to prophylactic treatment of a cancer disease, e.g., in an individual suspected to have the cancer disease, or at risk for developing the cancer disease. Prevention may include, but is not limited to, preventing or delaying onset or progression of the cancer disease and/or maintaining one or more symptoms of the cancer disease at a desired or sub-pathological level. The term “prevent” does not require the 100% elimination of the possibility or likelihood of occurrence of the event. Rather, it denotes that the likelihood of the occurrence of the event has been reduced in the presence of a composition or method as described herein. More particular, “prevent” or “preventing” refers to a decrease in the risk of occurrence of a cancer disease or symptom in a patient. As indicated above, the prevention may be complete, i.e., no detectable symptoms or disease, or partial, such that fewer symptoms or less severity of the disease are observed than would likely occur absent treatment.

The term “PSAT” refers to phosphoserine aminotransferase having the UniProtKB accession number Q9Y617, catalyzes the conversion of 3-phosphohydroxypyruvate (3P-Pyr) to 3-phosphoserine (3P-Ser).

The term “PSPH” refers to phosphoserine phosphatase having the UniProtKB accession number P78330, catalyzes the last step in the biosynthesis of serine, the conversion of 3-phosphoserine (3P-Ser) to serine.

As used herein, the term “serine” refers to an amino acid. An amino acid serine may be used in the biosynthesis of proteins (CAS Registry Number® is 56-45-1). Serine can be synthesized in the human body under normal physiological circumstances, making it a nonessential amino acid. It is the precursor to several amino acids including glycine (Registry Number 56-40-6) and cysteine. Within the disclosure, the term “significantly” used with respect to a change intends to mean that the observed change is noticeable and/or it has a statistic meaning.

As used herein, the term “subject” refers to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Preferably, the subject refers to a human. As used herein, “subject in need thereof” refers to a living organism suffering from or prone to a cancer disease or condition that can be treated by a method according to the present disclosure. Typically, a subject in need thereof according to the disclosure refers to any subject, preferably a human, afflicted with or susceptible to be afflicted with a cancer exhibiting recruitment of MDM2 to chromatin, in particular liposarcoma. Typically, a subject according to the present disclosure refers to any subject, preferably a human, afflicted with or susceptible to be afflicted with a liposarcoma. In some embodiments, the term “subject” refers to any subject afflicted with or susceptible to be afflicted with cancer types exhibiting recruitment of MDM2 to chromatin, including liposarcoma, ovarian cancers, glioblastoma, breast cancers, melanoma, colorectal cancers, kidney cancers, bone cancer, brain cancer, skin cancer, malignant hemopathies, AML (Acute myeloid leukemia), pancreatic cancer, prostate cancer, and lung cancer or cancer exhibiting exacerbated serine and glycine metabolism, including advanced melanoma and lung cancer.

By a “therapeutically effective amount” of the IL-6 signaling inhibitor of the present disclosure is meant a sufficient amount of the IL-6 signaling inhibitor for treating cancer at a reasonable benefit/risk ratio applicable to any medical treatment. The specific amount that is therapeutically effective can be readily determined by an ordinary medical practitioner and may vary depending on factors such as the type and stage of pathological processes considered, the subject's medical history and age, and the administration of other therapeutic agents. The specific therapeutically effective dose level for any particular subject will depend upon a variety of factors including the disorder being treated and the severity of the disorder; activity of the specific inhibitor employed; the specific composition employed, the age, body weight, general health, sex and diet of the subject; the time of administration, route of administration, and rate of excretion of the specific inhibitor employed; the duration of the treatment; drugs used in combination or coincidential with the specific inhibitor employed; and like factors well known in the medical arts. For example, it is well within the skill of the art to start doses of the inhibitor at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions may contain 0.01, 0.05, 0.1, 0.5, 1.0, 2.5, 5.0, 10.0, 15.0, 25.0, 50.0, 100, 250 or 500 mg of IL-6 signaling inhibitor of the present disclosure for the symptomatic adjustment of the dosage to the subject to be treated. A medicament typically contains from about 0.0002 mg to about 500 mg of the IL-6 signaling inhibitor of the present disclosure, preferably from 0.01 mg to about 100 mg of the IL-6 signaling inhibitor of the present disclosure. An effective amount of the IL-6 signaling inhibitor is ordinarily supplied at a dosage level from about 0.0002 mg/kg to about 500 mg/kg of body weight per day, especially from about 0.01 mg/kg to about 100 mg/kg, and in particular from about 0.1 mg/kg to 50 mg/kg of body weight per day.

The terms “treat” or “treatment” or “therapy” in the present disclosure refer to the administration or consumption in a subject in need thereof of an IL-6 signaling inhibitor or a pharmaceutical composition comprising an IL-6 signaling inhibitor of the present disclosure, optionally in combination with a serine/glycine deprivation of cancer cells according to the present disclosure, with the purpose to cure, heal, alleviate, relieve, alter, remedy, ameliorate, improve, or affect a cancer disorder as described herein, the symptoms of the condition, or to prevent or delay the onset of the symptoms, complications, or otherwise arrest or inhibit further development of the cancer disorder. More particularly, “treating” or “treatment” includes any approach for obtaining beneficial or desired results in a subject's cancer condition. The treatment may be administered to a subject having a cancer exhibiting recruitment of MDM2 to chromatin or who ultimately may acquire the cancer exhibiting recruitment of MDM2 to chromatin. Beneficial or desired clinical results can include, but are not limited to alleviation or amelioration of one or more cancer symptoms or conditions, diminishment or reduction of the extent of a cancer disease or of a cancer symptom, stabilizing, i.e., not worsening, the state of a cancer disease or of a cancer symptom, prevention of a cancer disease or of a cancer symptom's spread, delay or slowing of cancer disease or cancer symptom progression, amelioration or palliation of the cancer disease state, diminishment of the reoccurrence of cancer disease, and remission, whether partial or total and whether detectable or undetectable. In other words, “treatment” as used herein includes any cure, amelioration, or reduction of a cancer disease or symptom. A “reduction” of a symptom or a disease means decreasing of the severity or frequency of the disease or symptom, or elimination of the disease or symptom.

The list of sources, ingredients and components set forth in the present disclosure are understood to be described such that all combinations and mixtures thereof are also contemplated within the scope of the present disclosure.

It is understood that each maximum numerical limitation given in the disclosure encompasses each lower numerical limitation, as if such lower numerical limitations were expressly written. Each minimum numerical limitation given throughout the description encompasses each higher numerical limitation, as if such higher numerical limitations were expressly written herein. Each numerical range given throughout the present disclosure encompasses each narrower numerical range included within such wider numerical range, as if such narrower numerical ranges were all expressly written therein.

It may be referred to trade names of components comprising various ingredients used in the present description. The present disclosure does not intend to be limited to materials under any particular trade name. Equivalent materials (e.g., those obtained from a different source under a different name or reference number) to those indicated herein by trade name may be substituted and used in the present disclosure.

IL-6 Signaling Pathway: IL-6 and its Interaction with IL-6R, gp130 and STAT3

IL-6 is the founding member of the IL-6 cytokine family, which also includes IL-11, IL-27 p28/IL-30, IL-31, Leukemia inhibitory factor (LIF), Oncostatin M (OSM), Cardiotrophin-like cytokine (CLC), Ciliary neurotrophic factor (CNTF), Cardiotrophin-1 (CT-1), and Neuropoietin. IL-6 contains four long alpha-helical chains that are arranged in an up-up-down-down topology. IL-6 is produced by numerous different cell types and plays a critical role in regulating the acute phase response, inflammation, hematopoiesis, liver regeneration, metabolic control, bone metabolism, and, in particular, cancer progression.

IL-6 signaling occurs by the interaction between IL-6 to the membrane-bound form of the IL-6-specific receptor alpha (IL-6 R alpha or IL-6 R), which triggers its association with gp130, a transmembrane receptor protein that transduces the signals from IL-6 to STAT3, which then activates transcription of various genes. Association of gp130 with the IL-6/IL-6 R complex promotes gp130 dimerization and formation of a signaling complex consisting of IL-6, IL-6 R, and gp130. The signaling complex contains two copies of IL-6, two copies of the IL-6Ralpha, and two copies of gp130. Specifically, IL-6 binds to the gp130 receptor through three conserved epitopes known as sites I, II, and III. IL-6 must first form a complex with IL-6Ralpha through site I. Site II is a composite epitope formed by the binary complex of IL-6 and IL-6 R alpha, which interacts with cytokine binding region CHR and D2D3 of gp130. Subsequently, site III interacts with the gp 130 immunoglobulin-like activation domain (D1 or IGD) to form the signaling complex (Boulanger et al., (2003) Science, 300:2101). The site I binding epitope of IL-6 is localized to the A and D helices and interacts with IL-6 R alpha. The remaining four unique protein-protein interfaces in the signaling complex can be separated into two composite sites, sites II and III. Formation the signaling complex leads to the activation of multiple intracellular signaling pathways including the Jak-STAT pathway, the Ras-MAPK pathway, the p38 and JNK MAPK pathways, the PI 3-K-Akt pathway, and the MEK-ERK5 pathway (FIG. 12).

In particular, the mechanism of the activation of the Jak-STAT pathway comprises the activation of STAT3 by phosphorylation of a critical tyrosine residue mediated by growth factor receptor tyrosine kinases, Janus kinases, and/or the Src family kinases, etc. These kinases include but not limited to EGFR, JAKs, Abl, KDR, c-Met, Src, and Her2 [1]. Upon tyrosine phosphorylation, STAT3 forms homo-dimers and translocates to the nucleus, binds to specific DNA-response elements in the promoters of the target genes, and induces gene expression (Zhuang, Shougang (2013). Cellular Signalling. 25(9): 1924-1931).

According to some embodiments, IL-6 promotes cellular responses through a receptor complex consisting of at least one subunit of the signal-transducing glycoprotein gp130 and the IL-6 receptor (“IL-6R”). The IL-6R may also be present in a soluble form (“sIL-6R”). IL-6 binds to IL-6R, obtaining an IL-6/IL-6R complex, which then dimerizes the signal-transducing receptor gp130.

IL-6 signaling pathway is notably (but not exclusively) expressed in chondrocytes, endothelial cells, epithelial cells, fibroblasts, monocytes, macrophages, myocytes, osteoblasts, smooth muscle cells, synoviocytes and T cells.

The inventors have surprisingly found that a biological effect of IL-6 in myocytes may be to induce serine synthesis. In particular, it has been found that myocyte cells of a subject suffering from cancer exhibiting recruitment of MDM2 to chromatin, e.g. liposarcoma, synthesize serine. IL-6 is therefore produced by IL-6-expressed cancer cells exhibiting recruitment of MDM2 to chromatin, such as liposarcoma cells. Then IL-6 protein binds to IL-6 receptor and gp130 on the surface of the myocyte cells, to initiate the IL-6 signaling pathway, through the Jak/STAT3 pathway, in myocytes to allow serine synthesis as described above and illustrated in FIG. 12.

### IL-6 Signaling Inhibitors

An aspect of the present disclosure relates to IL-6 signaling inhibitors that block the biological effects of IL-6 on serine-producing cells, in particular myoblast cells.

“IL-6 signaling inhibitors” of the present disclosure encompass compounds selected in the group comprising (i) compounds blocking the interaction between IL-6 and IL-6 receptor, such as compounds that selectively block IL-6 recruitment to its IL-6 receptor with the consequence to inhibit the expression of genes involved in serine metabolism including phosphoglycerate dehydrogenase (PHGDH), phosphohydroxythreonine aminotransferase (PSAT) and phosphoserine phosphatase (PSPH), (ii) compounds blocking the interaction between IL-6 and gp130, (iii) compounds blocking the interaction between IL-6 receptor and gp130, (iv) compounds blocking the interaction between IL-6/IL-6R complex with gp130, and (v) compounds blocking the phosphorylation of STAT3 after the activation of the receptor of IL-6.

In some embodiments, IL-6 signaling inhibitors of the present disclosure may be selected from the group comprising IL-6 inhibitors, IL-6 receptor inhibitors, IL-6/IL-6R complex inhibitors, STAT3 inhibitors and gp130 inhibitors. “IL-6 signaling inhibitors” of the present disclosure include aptamers, antibodies and portions thereof; human or humanized forms of antibodies, modified antibodies, functional equivalents of antibodies, antisense oligonucleotides, siRNAs, shRNAs, ribozymes, or other proteins and molecules capable of binding and block interaction between either IL-6, IL-6R, IL-6/IL-6R complex, STAT 3 and/or gp130.

### Antibody

In some preferred embodiments, the IL-6 signaling inhibitor of the disclosure may be an antibody, or an antigen-binding portion thereof, directed against IL-6, IL-6 receptor, STAT3 and/or gp130.

In some embodiments of an antibody or portions thereof described herein, the antibody may be a monoclonal antibody, a polyclonal antibody, a multivalent antibody, or a chimeric antibody.

Antibodies are prepared according to conventional methodology.

The antibodies specified herein also encompass monoclonal antibodies. Monoclonal antibodies may be generated using the method of Kohler and Milstein (Nature, 256:495, 1975). To prepare monoclonal antibodies useful in the disclosure, a mouse or other appropriate host animal is immunized at suitable intervals (e.g., twice-weekly, weekly, twice-monthly or monthly) with antigenic forms of IL-6, IL-6 receptor, IL-6/IL-6R complex, STAT3 and/or gp130. The mouse may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal administration or other routes. A given animal may be immunized with multiple forms of the antigen by multiple routes.

Briefly, the antigen may be provided as synthetic peptides corresponding to antigenic regions of interest in IL-6, IL-6 receptor, IL-6/IL-6R complex, STAT3 and/or gp130. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.c., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modem Immunology Wiley & Sons, Inc., New York; Roitt, I. (1991) Essential Immunology, 7th Ed., Blackwell Scientific Publications, Oxford). The Fc′ and Fc regions, for instance, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

The monoclonal antibodies specified herein also encompass humanized anti-antibodies, in particular humanized anti-IL-6 antibodies or anti-IL-6 receptor antibodies or anti-gp130 antibodies or a STAT3 antibody. “Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al, Nature 321:522-525 (1986); Riechmann et al., Nature 332:323-329 (1988); and Presta, Curr. Op. Struct. Biol. 2:593-596 (1992).

The monoclonal antibodies specified herein further encompass human antibodies, in particular human anti-IL-6 antibodies, human anti-IL-6 receptor antibodies, human anti-STAT3 antibodies and/or human anti-gp130 antibodies. A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In some embodiments, the human antibody is selected from a phage library, where that phage library expresses human antibodies (Vaughan et al. Nature Biotechnology 14:309-314 (1996): Sheets et al. Proc. Natl. Acad. Sci. 95:6157-6162 (1998)); Hoogenboom and Winter, J. Mol. Biol, 227:381 (1991); Marks et al., J. Mol. Biol, 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Fishwild et al., Nature Biotechnology 14:845-51 (1996); Neuberger, Nature Biotechnology 14:826 (1996). Alternatively, the human antibody may be prepared via immortalization of human B lymphocytes producing an antibody directed against a target antigen (such B lymphocytes may be recovered from an individual or may have been immunized in vitro). See, e.g., Boerner et al., J. Immunol, 147 (1):86-95 (1991); and U.S. Pat. No. 5,750,373. Humanized antibodies may be produced by obtaining nucleic acid sequences encoding CDR domains and constructing a humanized antibody according to techniques known in the art. Methods for producing humanized antibodies based on conventional recombinant DNA and gene transfection techniques are well known in the art (See, e.g., Riechmann L. et al. 1988; Neuberger M S. et al. 1985). Antibodies can be humanized using a variety of techniques known in the art including, for example, CDR-grafting (PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see EP 125,023 and WO 96/02576).

Thus, as will be apparent to one of ordinary skill in the art, the present disclosure also provides for F(ab′), 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non human sequences. The present disclosure also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the present disclosure is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. The term “VHH” refers to the single heavy chain having 3 complementarity determining regions (CDRs): CDR1, CDR2 and CDR3. The VHH according to the disclosure can readily be prepared by an ordinarily skilled artisan using routine experimentation. VHHs or sdAbs are usually generated by PCR cloning of the V-domain repertoire from blood, lymph node, or spleen cDNA obtained from immunized animals into a phage display vector, such as pHEN2. For instance, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In some embodiments, the IL-6 signaling inhibitor of the disclosure may be an aptamer. Aptamers are nucleic acid molecules having specific binding affinity to molecules that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by Exponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications uses and advantages of this class of molecules have been reviewed in Jayasena S.D., 1999. Peptide aptamers consists of a conformationally constrained antibody variable region displayed by a platform protein, such as E. coli Thioredoxin A that are selected from combinatorial libraries by two hybrid methods (Hamdi et al., 2011). Then after raising aptamers directed against IL-6, IL-6 receptor, IL-6/IL-6R complex, STAT3 and/or gp130 as above described, the skilled artisan can easily select those inhibiting IL-6 signaling pathway.

In some embodiments, the IL-6 signaling inhibitor of the present disclosure may be a bicyclic peptide.

Bicyclic polypeptides are peptides containing three cysteine residues and two regions of six random amino acids (Cys-(X)₆-Cys-(X)₆-Cys), wherein Cys is cysteine and X is any of the 20 proteinogenic amino acids, displayed on phage and cyclised by covalently linking the cysteine side chains to a small molecule scaffold (tris-(bromomethyl) benzene (TBMB)). Bicyclic polypeptides may be prepared according to conventional methodology described in WO2009/098450 and Heinis et al., Nat Chem Biol 5, 502-507 (2009).

In some embodiments, the bicyclic polypeptide targets antigens of interest, in particular an IL-6 signaling pathway antigen, more particularly an IL-6, an IL-6R, a STAT 3 protein, an IL-6/IL-6R complex or a gp130.

In some embodiments, the bicyclic polypeptide as described in the present disclosure have specific utility as high affinity binders of IL-6, IL-6R, IL-6/IL-6R complex, STAT3 or gp130.

In some embodiments, the IL-6 signaling inhibitor may be a blocking anti-IL-6 antibody, a blocking anti-IL-6 receptor antibody, a blocking anti-IL-6/IL-6R complex antibody, a STAT3 inhibitor and/or an anti-gp130 compound.

In some preferred embodiments, the IL-6 signaling inhibitor of the present disclosure may be a blocking anti-IL-6 antibody, preferably a blocking monoclonal anti-IL-6 antibody.

Tests and assays for determining whether a compound is a blocking anti-IL-6 antibody are well known by the skilled person in the art such as described in Wijdenes et al., Mol Immunol. 1991. Some of those blocking anti-IL-6 antibodies are illustrated by Xu et al., British Journal of Clinical Pharmacology, 72:270-281, Rossi et al., Br J Cancer. 2010 Oct. 12; 103(8):1154-62, WHO Drug Information Vol. 24, No. 2, 2010, Proposed INN: List 103, or Mease et al., Arthritis & Rheumatology, 68:2163-2173.

In some embodiments, a blocking anti-IL-6 antibody is selected from the group comprising sirukumab, siltuximab, olokizumab and clazakizumab.

In some embodiments, the IL-6 signaling inhibitor of the present disclosure may be a blocking anti-IL-6 receptor antibody, preferably a blocking monoclonal anti-IL-6 receptor antibody.

Tests and assays for determining whether a compound is a blocking anti-IL-6 receptor antibody are well known by the skilled person in the art. Some of those blocking anti-IL-6 receptor antibody are illustrated by Nishimoto et al., Ann Rheum Dis. 2009 October; 68(10):1580-4 or Genovese et al., RMD Open. 2019 Aug. 1; 5(2):e000887.

In some embodiments, a blocking anti-IL-6 receptor antibody is selected from the group comprising tocilizumab, sarilumab, and TZLS-501.

In some preferred embodiments, the IL-6 signaling inhibitor of the present disclosure may be a blocking anti-IL-6/IL-6R complex antibody, preferably a blocking monoclonal anti-IL-6/IL-6R complex antibody.

Some of those blocking anti-IL-6/IL-6R complex antibodies are illustrated by, for instance, U.S. Pat. No. 10,759,862.

In some embodiments, a blocking anti-IL-6/IL-6R complex antibody may be TZLS-501.

### Anti-Sense Oligonucleotides

In some embodiments, the IL-6 signaling inhibitor of the invention may be an IL-6 expression inhibitor, an IL-6 receptor expression inhibitor, a STAT3 expression inhibitor and/or a gp130 expression inhibitor.

In some embodiments, expression inhibitors for use in the method according to the present disclosure may be anti-sense oligonucleotides.

In some embodiments, the IL-6 signaling inhibitor of the present disclosure may be an anti-sense oligonucleotide, in particular an anti-sense oligonucleotide directed to the gene or mRNA coding for IL-6, IL-6 receptor, STAT3 or gp130.

The anti-sense oligonucleotides have the biological effect to inhibit the expression of a gene, in particular a gene coding for the expression of IL-6, IL-6 receptor, STAT3 or gp130. Anti-sense oligonucleotides, including anti-sense RNA molecules, such as siRNAs and shRNAs, and anti-sense DNA molecules, would act to directly block the translation of mRNA by binding thereto and thus preventing protein translation or increasing mRNA degradation, thus decreasing the level of proteins, and thus activity, in a cell. For example, antisense oligonucleotides of at least about 15 bases and complementary to unique regions of the mRNA transcript sequence encoding IL-6, IL-6 receptor, STAT3 and/or gp 130 can be synthesized, e.g., by conventional phosphodiester techniques and administered by e.g., intravenous injection or infusion. Methods for using anti-sense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Further, anti-sense oligonucleotides may be a single guide RNA (sgRNA). A custom sgRNA is used in the CRISPR/Cas9 system. CRISPR/Cas9 is a flexible gene editing tool, allowing the genome to be manipulated in diverse ways. For instance, CRISPR/Cas9 has been successfully used to knockout genes, knock-in mutations, overexpress or inhibit gene activity, and provide scaffolding for recruiting specific epigenetic regulators to individual genes and gene regions. A custom single guide RNA (sgRNA) contains a targeting sequence (crRNA sequence) and a Cas9 nuclease-recruiting sequence (tracrRNA). The crRNA region is a 20-nucleotide sequence that is homologous to a region in the target gene, in particular IL-6 gene, IL-6R gene, STAT3 gene or gp130 gene, and will direct Cas9 nuclease activity (see e.g. Gilbert et al., Cell. 2013 Jul. 18; 154(2):442-51, Platt et al., Cell. 2014 Oct. 9; 159(2):440-55)

In some embodiments, an anti-sense oligonucleotide according to the present disclosure may be a siRNA, a shRNA or a sgRNA,

In some preferred embodiments, an anti-sense oligonucleotide according to the present disclosure is a shRNA.

As shown in FIG. 3, small inhibitory RNAs (siRNAs) and short hairpin RNAs (shRNAs) may also function as IL-6 signaling expression inhibitors in the present disclosure. siRNAs and shRNAs of the present disclosure are double-stranded RNAs or modified RNA molecules which down-regulates or silences (prevents) the expression of a gene of its endogenous (cellular) counterpart. Gene expression can be reduced by contacting the subject or cell with a small double stranded RNA (dsRNA) as defined herein, or a vector or construct causing the production of a small double stranded RNA, such that IL-6, IL-6 receptor, STAT3 and/or gp130 expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, GJ. (2002); McManus, MT. et al. (2002); Brummelkamp, TR. et al. (2002); U.S. Pat. Nos. 6,573,099 and 6,506,559; and International Patent Publication Nos. WO 01/36646, WO 99/32619, and WO 01/68836).

In some embodiments, an anti-sense oligonucleotide according to the present disclosure comprises or consists of nucleic acids having a sequence with at least about 80%, 85%, 86%, 87%, 88%, 89%, 90%, 91%, 92%, 93%, 94%, 95%, 96%, 97%, 98%, 99%, or 100% identity with the full-length nucleic acids of the target gene, in particular IL-6 gene, IL-6R gene, STAT3 gene and gp130 gene, or a portion thereof.

IL-6 gene refers to the nucleic acids having the sequence illustrated by NCBI Gene ID: 3569.

IL-6R gene refers to the nucleic acids having the sequence illustrated by NCBI Gene ID: 3570.

Gp130 gene refers to the nucleic acids having the sequence illustrated by NCBI Gene ID: 16195.

STAT3 gene refers to the nucleic acids having the sequence illustrated by NCBI Gene ID: 6774.

In certain other embodiments, an anti-sense oligonucleotide according to the present disclosure comprises or consists of at least about 15 contiguous nucleotides, in particular at least about 15, 16, 17, 18, or 19 contiguous nucleotides, of a sequence that is identical to the target sequence, in particular IL-6, IL-6R, STAT3 and gp130, or a portion thereof.

In preferred embodiments, an anti-sense oligonucleotide according to the present disclosure is capable of mediating target-specific anti-sense oligonucleotide silencing IL-6 expression, IL-6R expression, gp130 expression or STAT3 expression.

In some embodiments, the IL-6 signaling inhibitor of the present disclosure may be a shRNA directed to IL-6, in particular directed to IL-6 gene.

In some embodiments, the IL-6 signaling inhibitor of the present disclosure may be a shRNA directed to IL-6 receptor, in particular directed to IL-6R gene.

In some embodiments, the IL-6 signaling inhibitor of the present disclosure may be a shRNA directed to gp13, in particular directed to gp130 gene.

In some embodiments, the IL-6 signaling inhibitor of the present disclosure may be a shRNA directed to STAT3, in particular directed to STAT3 gene.

Ribozymes can also function as IL-6 signaling expression inhibitors in the present disclosure. Ribozymes have also the biological effect to inhibit the expression of a gene, in particular a gene coding for the expression of IL-6, IL-6 receptor, STAT3 or gp130. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage.

Engineered hairpin or hammerhead motif ribozyme molecules that specifically and efficiently catalyze endonucleolytic cleavage of mRNA sequences are thereby useful within the scope of the present disclosure. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Anti-sense oligonucleotides and ribozymes useful as IL-6 signaling expression inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the disclosure can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-0-methyl rather than phosphodiesterase linkages within the oligonucleotide backbone.

Anti-sense oligonucleotides, and ribozymes of the present disclosure may be delivered in vivo alone or in association with a vector. In its broadest sense, a “vector” is any vehicle capable of facilitating the transfer of the anti-sense oligonucleotide or ribozyme nucleic acid to the cells, preferably cancer cells expressing IL-6, IL-6 receptor, STAT3 or gp130. Preferably, the vector transports the nucleic acid to cells with reduced degradation relative to the extent of degradation that would result in the absence of the vector. In general, the vectors useful in the present disclosure include, but are not limited to, plasmids, phagemids, viruses, other vehicles derived from viral or bacterial sources that have been manipulated by the insertion or incorporation of the anti-sense oligonucleotide or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors may be based on non-cytopathic eukaryotic viruses in which non-essential genes have been replaced with the gene of interest, the adeno-viruses and adeno-associated viruses.

Other preferred vectors may be plasmid vectors. Plasmid vectors have been extensively described in the art and are well known to those of skill in the art. See e.g., SANBROOK et al, “Molecular Cloning: A Laboratory Manual,” Second Edition, Cold Spring Harbor Laboratory Press, 1989.

### Chemical Compound

In some embodiments, the IL-6 signaling inhibitor of the present disclosure may be a chemical compound.

In some embodiments, a chemical compound of the present disclosure includes compounds inhibiting the IL-6 expression, IL-6R expression, STAT expression, STAT activation, the phosphorylation of STAT3 and/or gp 130 expression.

In some particular embodiments, a chemical compound of the present disclosure may be a blocking anti-gp130 compound, a compound that inhibits the phosphorylation of STAT3, a compound that inhibits the activation of STAT3 or a compound that inhibit the expression of STAT3.

Some of those blocking anti-gp130 compound are illustrated by, for instance, Wu et al., Mol Cancer Ther. 2016 November; 15(11):2609-2619.

In some embodiments, a gp-130 inhibitor of the present disclosure may be bazedoxifene,

In some embodiments, a STAT3 inhibitor may be selected from SH5-07, APTSTAT3-9R, C188-9, BP-1-102, Niclosamide (BAY2353), STAT3-IN-1, WP1066, Cryptotanshinone, Stattic, Resveratrol (SRT501), S31-201, HO-3867, Napabucasin (BBI608), Brevilin A, Artesunate (WR-256283), Bosutinib (SKI-606), TPCA-1, SC-43, Ginkgolic acid C17: 1, Ochromycinone (STA-21), Colivelin, Cucurbitacin IIb, GYY4137, Scutellarin, Kaempferol-3-O-rutinoside, Cucurbitacin I, SH-4-54, Nifuroxazide and Pimozide.

In some embodiments, a STAT3 inhibitor may be C188-9, Stattic or a combination thereof.

As used herein “bazedoxifene” has its general meaning in the art and consists of a gp130 inhibitor having the formula: 1-[[4-[2-(Hexahydro-1H-azepin-1-yl)ethoxy]phenyl]methyl]-2-(4-hydroxyphenyl)-3-methyl-1H-indol-5-ol monoacetate (salt) with the molecular formula C30H34N2O3·C2H4O2 and the CAS Number 198481-33-3.

As used herein “C188-9” is a potent inhibitor of STAT3 that binds to STAT3 with high affinity. C188-9 may be defined by the reference: CAS No. 432001-19-9.

As used herein “Stattic” is a small molecule inhibiting STAT3 activation. Stattic may be defined by the reference: CAS No. 19983-44-9.

IL-6 signaling inhibitors of the disclosure may be effective in the prevention and/or treatment of a cancer exhibiting recruitment of MDM2 to chromatin, in particular liposarcoma. IL-6 signaling inhibitors of the disclosure may be used in combination with another therapeutic agent, in particular with a serine/glycine deprivation diet.

Alone or in combination with other therapeutic agents, IL-6 signaling inhibitors of the disclosure are used at a therapeutically effective amount. The effective therapeutic amount may vary depending on several factors such as the nature and status of the cancer disease to be treated, the age, gender, weight of the patient, the concomitant presence of other diseases, the diet, the concomitant presence of other treatment. It is upon the skilled person to determine the appropriate therapeutically effective amount of an IL-6 signaling inhibitor of the disclosure, depending on those factors and other well-known in the art.

A combination of an IL-6 signaling inhibitor of the present disclosure with a serine/glycine deprivation diet results in a synergistic effect. A synergistic effect of two agents, such as an IL-6 signaling inhibitor disclosed herein and a serine/glycine deprivation diet, corresponds to a situation in which the total effect of the combination is greater than the sum of the individual effect.

According to some embodiments, in particular when used in combination with a serine/glycine deprivation diet, an IL-6 signaling inhibitor of the present disclosure may be administered to a subject at a dosage from about 0.0002 mg/kg to about 500 mg/kg of body weight per day.

According to some embodiments, in particular when used in combination with a serine/glycine deprivation diet, an IL-6 signaling inhibitor of the present disclosure may be administered to a subject at a dosage from about 0.0002 mg/kg to about 300 mg/kg of body weight per day.

According to some embodiments, in particular when used in combination with a serine/glycine deprivation diet, an IL-6 signaling inhibitor of the present disclosure may be administered to a subject at a dosage from about 0.001 mg/kg to about 200 mg/kg of body weight per day.

According to some embodiments, in particular when used in combination with a serine/glycine deprivation diet, an IL-6 signaling inhibitor of the present disclosure may be administered to a subject at a dosage from about 0.01 mg/kg to about 100 mg/kg of body weight per day.

According to some embodiments, in particular when used in combination with a serine/glycine deprivation diet, an IL-6 signaling inhibitor of the present disclosure may be administered to a subject at a dosage from about 0.1 mg/kg to about 50 mg/kg of body weight per day.

Particularly the IL-6 signaling inhibitor may be administered at a dosage of about 0.001, 0.005, 0.01, 0.05, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0 mg/kg of body weight per day.

Illustratively, for a 70-kilogram adult human, it may be administered an IL-6 signaling inhibitor at a dose in the range of about 0.014 mg (milligram) to about 35 g (gram) per day, or with a dose in the range of about 0.7 mg to about 7 g, or preferably a dose of about 7 mg to about 3.5 g is most preferred.

It is intended that a skilled person knows how to adjust the dosage of IL-6 signaling inhibitors depending on the route of administration, the weight, age, gender of the patient to be treated, as well as depending on possible preexisting conditions to consider, and possible additional treatment administered.

For instance, when treating a solid tumor cancer cells in a subject, the effectiveness of a given dose may be evaluated as follows. At various points after administration of an IL-6 signaling inhibitor of the disclosure, solid or liquid biopsies are withdrawn from the subject in need thereof and tested for IL-6 signaling activation, for example by immunostaining with an appropriate anti-phosphotyrosine antibody. The goal is to have continuous, essentially complete inhibition of IL-6 signaling activation in the tumor cancer cells. If inhibition of IL-6 signaling activation is not complete, the dose may be increased, or the dosage frequency may be increased.

All or part of the particular features and embodiments relating to the IL-6 signaling inhibitors and compositions comprising it according to the present disclosure, also apply to the intended uses and methods of the present disclosure.

### Serinelglycine Source Deprivation and Diet

The methods as described herein may include two treatments wherein one is the stand-alone administration of an IL-6 signaling pathway inhibitor and the other is the administration of an IL-6 signaling pathway inhibitor in combination with a serine and glycine deprivation of cancer cells exhibiting recruitment of MDM2 to chromatin.

As previously mentioned herein, IL-6 signaling inhibitors of the present disclosure are implemented in methods for the treatment and prevention of cancer, in particular in combination with a serine/glycine deprivation of cancer cells.

As used herein, a cancer cell deprived of serine/glycine means that the environment of the cancer cell fails to provide an adequate supply of serine and/or glycine needed to allow a normal development of the tumor in the subject.

In some embodiments of the present disclosure the combination of the two treatments of the present disclosure involves a drastic or reduced serine depletion of cancer cells.

In some embodiments, the methods of the present disclosure may be implemented on cancer cells exhibiting recruitment of MDM2 to chromatin that are deprived of serine and glycine.

A serine and glycine deprivation of cancer cells may comprise any methods known by the skilled person that avoid or limit the entry of a serine or glycine molecule within a cancer cell. In some embodiments, a serine and glycine deprivation of cancer cells of a subject may comprise a deprivation or reduction of any source of serine and/or any source of glycine in a subject, an administration of a serine/glycine-deprived diet to a subject, or an inhibition of the expression of serine and glycine receptors on cancer cells of a subject.

In some embodiments, cancer cells of a subject in need thereof may be deprived of serine and glycine. In some embodiments, a subject in need thereof may be fed with a serine/glycine-deprived diet. In some embodiments, the subject may be fed with a unique serine/glycine-deprived diet during a certain period of treatment.

A serine/glycine-deprived diet of the present disclosure may be chemically synthetized.

A serine/glycine-deprived diet of the present disclosure may comprise a wide variety of components. Non-limiting examples of components that can be incorporated in the serine/glycine-deprived diet may be selected from: free amino acids, carbohydrates, fatty acids, water, crude fat, crude fibers, NFE, ash, minerals, vitamins, oligo-elements, electrolytes, or condiments.

In some embodiments, a serine/glycine-deprived diet of the present disclosure may comprise carbohydrates, water, vitamins, oligo-elements, and electrolytes.

Carbohydrate comprised in the serine/glycine-deprived diet of the present disclosure encompasses a mixture of polysaccharides and sugars. Carbohydrates can be supplied under the form of any of a variety of carbohydrate sources known by those skilled in the art, including starch (any kinds, corn, wheat, barley, etc.) beet pulp (which contain a bit of sugars), and psyllium.

Vitamins comprised in the serine/glycine-deprived diet of the present disclosure may encompass vitamin A, vitamins B, vitamin C, vitamin D, vitamin E, vitamin K or a mixture thereof. Vitamins B encompass vitamin B1, vitamin B2, vitamins B3 (PP), vitamin B5, vitamin B6, vitamin B8, vitamin B9, vitamin B12, or a mixture thereof.

Illustratively, a source of vitamins may be the CERNEVIT composition which comprises vitamin A, vitamin B1, vitamin B2, vitamin B5, vitamin B6, vitamin B8, vitamin B9, vitamin B12, vitamin C, vitamin D, vitamin E and vitamin B3.

Oligo-elements comprised in the serine/glycine-deprived diet of the present disclosure may encompass arsenic, bore, chlore, chrome, cobalt, copper, iron, fluor, iodine, lithium, manganese, molybdenum, nickel, selenium, silicon, sulfur, vanadium, zinc, or a mixture thereof.

Illustratively, a source of oligo-elements may be the NUTRYELT composition which comprises iron, copper, manganese, zinc, fluor, iodine, selenium, chrome and molybdenum.

Electrolytes comprised in the serine/glycine-deprived diet of the present disclosure may encompass potassium, sodium, calcium, magnesium, chloride, phosphorus, salt thereof, or a mixture thereof.

Illustratively, electrolytes which may be present in the serine/glycine-deprived diet of the present disclosure may be NaCl and/or KCl.

In some embodiments, a serine/glycine-deprived diet of the present disclosure may further comprise additional components such as, antioxidants, chelating agents, osmolality modifiers, buffers, neutralization agents and the like that improve the stability, uniformity and/or other properties of the serine/glycine-deprived diet.

In some preferred embodiments, the serine/glycine-deprived diet according to the present disclosure may be administered to a subject for a period of 1 day to 15 days, more preferably for a period of at least 5 days to at most 10 days.

In some embodiments, a period of 1 day to 15 days encompasses 2 days, 3 days, 4 days, 5 days, 6 days, 7 days, 8 days, 9 days, 10 days, 11 days, 12 days, 13 days and 14 days.

Accordingly, the serine/glycine-deprived diet according to the present disclosure may be administered for a period of at most 10 days without any additional nutritional support excepted water.

According to some embodiments, when used alone or in combination with an IL-6 signaling inhibitor of the present disclosure, the serine/glycine-deprived diet according to the present disclosure may be administered to a subject at a dosage from about 20 mL/kg to about 50 mL/kg of body weight per day.

According to some embodiments, when used alone or in combination with an IL-6 signaling inhibitor of the present disclosure, the serine/glycine-deprived diet according to the present disclosure may be administered to a subject at a dosage from about 25 mL/kg to about 45 mL/kg of body weight per day.

According to some embodiments, when used alone or in combination with an IL-6 signaling inhibitor of the present disclosure, the serine/glycine-deprived diet according to the present disclosure may be administered to a subject at a dosage of about 30 mL/kg of body weight per day.

Particularly, the serine/glycine-deprived diet according to the present disclosure may be administered at a dosage of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50.0 mL/kg of body weight per day.

Illustratively, for a 70-kilogram adult human, it may be administered the serine/glycine-deprived diet of the disclosure at a dose in the range of about 1.4 L (liter) to about 3.5 L per day, or with a dose in the range of about 1.75 L to about 3.15 L, or preferably a dose of about 2.1 L (2100 mL) is most preferred.

It is intended that a skilled person knows how to adjust the dosage of serine/glycine-deprived diet depending on the route of administration, the weight, age, gender of the patient to be treated, as well as depending on possible preexisting conditions to consider, and possible additional treatment administered.

In some embodiments, the serine/glycine-deprived diet of the disclosure may be administered by infusion, subcutaneous, intradermal, intramuscular, or intraperitoneal injection, inhalation, or oral administration, in particular by infusion.

In some embodiments, the serine/glycine-deprived diet of the disclosure may be administered by infusion for a continuous intravenous injection or repeated discontinuous injections.

In certain embodiments, the serine/glycine-deprived diet of the disclosure may be administered to a subject once or twice a day.

In some embodiments, the administration of the serine/glycine-deprived diet of the disclosure may be repeated every month, every month, or more.

In some embodiments, cancer cells of a subject may have a limited supply of a source of serine and/or glycine.

In particular, a serine/glycine-deprived diet may be a feeding regime determined by the skilled person which may adapt the feeding regime each day of the subject in order to limit the intake of serine and/or glycine in the diet administered to the subject during a given period of treatment.

In some embodiments, a subject in need thereof subjected to a serine/glycine-deprived diet may receive a diet comprising a lower amount of serine and/or glycine than a normal diet with a sufficient amount of glycine and/or serine to maintain a good health of the subject.

Particularly, a sufficient amount of serine and/or glycine to maintain a good health of the subject means that it may be administered in a subject an amount of about 500 to 2000 mg per day of serine and/or glycine.

In some embodiments, a subject in need thereof subjected to a deprivation of serine and/or glycine may receive serine and/or glycine in an amount of about 1 500 mg/day or less, 1 000 mg/day or less, 500 mg/day or less.

In some embodiments, a subject in need thereof subjected to a deprivation of serine and/or glycine may receive serine and/or glycine in an amount of about 500 mg/day or less, 400 mg/day or less, 300 mg/day or less, 200 mg/day or less, 100 mg/day or less, or 50 mg/day or less.

It is intended that a skilled person knows how to adjust the amount of serine/glycine by the dosage of a source of serine and/or glycine in a diet depending on the the route of administration, the weight, age, gender of the patient to be treated, as well as depending on possible preexisting conditions to consider, and possible additional treatment administered.

All or part of the particular features and embodiments relating to the serine/glycine-deprived diet according to the present disclosure, also apply to the intended uses and methods of the present disclosure.

### Cancer Exhibiting Recruitment of MDM2 to Chromatin

The oncoprotein MDM2 is mainly known as a major negative regulator of the tumor suppressor p53. However, recent studies have indicated that MDM2 may have a p53-independent oncogenic activity (Cissé et al., Sci. Transl. 2020). In the context of the present disclosure, it is specifically targeted cancer cell lines wherein MDM2 is localized in the cell nucleus.

In the context of the present disclosure, a “cancer exhibiting recruitment of MDM2 to chromatin” means that MDM2 is localized in the cell nucleus.

Without wishing to be bound by any particular theory, it is specified that a cancer exhibiting recruitment of MDM2 to chromatin does not equate to cancer exhibiting a MDM2 overexpression in the cell. IA cancer exhibiting recruitment of MDM2 to chromatin encompass both (i) cancers exhibiting overexpression of MDM2 and (ii) cancers that do not exhibit MDM2 overexpression. A cancer exhibiting recruitment of MDM2 to chromatin may be a cancer wherein the cancer cells exhibit a MDM2 overexpression in the cytoplasm and wherein at least a part of the cellular MDM2 is localized in the cell nucleus. A cancer exhibiting recruitment of MDM2 to chromatin may be a cancer wherein MDM2 is not overexpressed in the cancer cells and wherein at least part of the cellular MDM2 is localized in the cell nucleus.

Accordingly, in some embodiments, a cancer exhibiting recruitment of MDM2 to chromatin may not be a cancer exhibiting a MDM2 overexpression in the cytoplasm.

It has been shown in the examples of the present disclosure that cancer cell lines expressing high level of IL-6 exhibit an overexpression of MDM2. In contrast, it has been shown that cancer cell lines exhibiting an overexpression of MDM2 do not necessarily express IL-6. Therefore, and without wishing to be bound by any particular theories, it is believed that cancer cells lines exhibiting recruitment of MDM2 to chromatin express a high level of IL-6.

A cancer exhibiting recruitment of MDM2 to chromatin may be diagnosis with, for instance, the method of diagnosis disclosed in WO2019106126 or in Cissé et al. (2020, Sci Trans Med, Vol. 12; 547).

In some embodiments, a cancer exhibiting recruitment of MDM2 to chromatin may be diagnosed in a subject with any method allowing to observe the localization of a protein, in particular MDM2, in a cancer cell sample or a cancer tissue sample.

Determination of a cancer exhibiting recruitment of MDM2 to chromatin in a subject may be also a mean of classifying a subject according to the type of cancer he is affected by. In particular, a subject may be classified as being affected with a cancer exhibiting a recruitment of MDM2 to chromatin or as being affected by a cancer that do not exhibit a recruitment of MDM2 to chromatin.

For instance, methods allowing to determine the localization of a protein, in particular MDM2, in a cancer cell sample or a cancer tissue sample may be made by immunofluorescence, in particular by microscopy or cytometry, or by immunohistochemistry.

In some particular embodiments, a method for determining cancers exhibiting recruitment of MDM2 to chromatin in a subject in need thereof, may comprise the steps of: a) providing a cancer cell sample obtained from said subject, b) determining, in particular by immunofluorescence or immunohistochemistry, the localization of MDM2 in cancer cells, and c) concluding that the subject have a cancer exhibiting recruitment of MDM2 to chromatin when MDM2 is localized at nucleus (and hence on chromatin) or concluding that the subject do not have a cancer exhibiting recruitment of MDM2 to chromatin when MDM2 is not localized in the nucleus, in particular when MDM2 is localized in the cytoplasm of the cancer cell.

Illustratively, the localization of MDM2 in cancer cells of a subject may be determined by immunohistochemistry. Cancer cells or tissues are collected from the tumor in the subject. The cancer cells are placed on a solid support. Then, an anti-MDM2 antibody is added to the cancer cells preparation. Subsequently, it is added a secondary antibody bound to a detection system (enzyme bound to the antibody, whose presence in the substrate causes a colored (peroxidase) or fluorescent (Rhodamine) reaction), causing a signal visible to the naked eye, or by microscopy and spectrophotometry techniques. Subsequently, it may be determined, through the observation of the signal color, whether MDM2 is localized at the nucleus (and hence on chromatin), or in the cytoplasm.

In some other embodiments, a method for diagnosing cancers exhibiting recruitment of MDM2 to chromatin in a subject in need thereof, may comprise the steps of:
i) determining the level of nuclear-bound MDM2 in the biological sample, in particular the level of chromatin-bound MDM2,
ii) concluding that the subject is affected by a cancer exhibiting recruitment of MDM2 to chromatin when the proportion of nuclear-bound MDM2 cells determined at step ii) represents more than about 1% of the cancer cells in the sample.

In some particular embodiments, a method for diagnosing cancers exhibiting recruitment of MDM2 to chromatin in a subject in need thereof, may comprise the steps of:
i) determining the level of nuclear-bound MDM2 in the cancer cells of the biological sample,
ii) concluding that the subject is affected by a cancer exhibiting recruitment of MDM2 to chromatin when the proportion of nuclear-bound MDM2 cells determined at step ii) represents more than about 1% of the cancer cells in the sample.

Assessing the level of nuclear-bound MDM2, in particular chromatin-bound MDM2, in cancer cells of a biological sample of a subject can be readily determined by a skilled person in the art.

The level of nuclear-bound MDM2, in a biological sample of a subject may be also determined in patient-derived tumor samples by immunoblot on cellular fractionation isolating chromatin

In some embodiments, a biological sample may be a tissue sample.

In particular, a liquid sample may be whole blood, plasma, or serum. Techniques for the collection of a liquid sample of a subject are well-known by a skilled person in the art.

In particular, a tissue sample may be a cancer tissue sample of the subject. Techniques for the collection of a tissue sample of a subject are well-known by a skilled person in the art. For instance, the collection of a tissue sample may be realized by a biopsy.

The methods of diagnosis described herein may be implemented as a biomarker test to determine if a subject is suffering from a cancer exhibiting recruitment of MDM2 to chromatin.

In some embodiments, the methods of diagnosis described herein provide clinical information. Illustratively, the methods of diagnosis as described herein allow completing information relating to a biopsy sample provided from a subject suffering from a cancer exhibiting recruitment of MDM2 to chromatin.

Illustratively, the methods of diagnosis described herein allow determining the presence of MDM2 to chromatin in a subject suffering from a cancer, wherein the detection of MDM2 to chromatin may permit the medical practitioner to decide to administer an IL-6 inhibitor of the present disclosure to the subject suffering from cancer. The medical practitioner may adapt the subject treatment by administering to the said subject an IL-6 inhibitor according to the present disclosure further with a serin/glycine deprived diet according to the present disclosure.

According to the present disclosure, a recruitment of MDM2 to chromatin in the cancer cells of a subject in need thereof, may occur only after a certain period subsequent to the occurrence of the cancer or in contrast may occur immediately upon the occurrence of the cancer. In particular, MDM2 recruitment to chromatin may occur at the onset of the cancer but also during the different phases of the cancer, including following administration to the said subject of at least a first treatment against cancer.

A cancer exhibiting recruitment of MDM2 to chromatin within the present disclosure may be selected from bone cancer, brain cancer, ovary cancer, breast cancer, lung cancer, colorectal cancer, osteosarcoma, skin cancer, malignant hemopathies, pancreatic cancer, prostate cancer and liposarcoma.

In some embodiments, a cancer exhibiting recruitment of MDM2 to chromatin considered within the present disclosure may be liposarcoma.

The term “liposarcoma” or “LPS” has its general meaning in the art and refers to soft tissue sarcomas of mesenchymal origin such as revised in the World Health Organisation Classification ICD10 C49.9. The term “liposarcoma” also refers to well-differentiated and de-differentiated liposarcoma (WD- and DD-LPS). The term “liposarcoma” also relates to Malignant mesenchymal neoplasms, a type of soft tissue sarcoma, a group of lipomatous tumors of varying severity ranging from slow-growing to aggressive and metastatic. Liposarcomas are most often located in the lower extremities or retroperitoneum, but they can also occur in the upper extremities, neck, peritoneal cavity, spermatic cord, breast, vulva and axilla. The term “liposarcoma” also relates to dedifferentiated liposarcoma and well-differentiated liposarcoma.

In some embodiments, the term “liposarcoma” refers to liposarcoma exhibiting recruitment of MDM2 to chromatin.

All or part of the particular features and embodiments relating to cancer exhibiting recruitment of MDM2 to chromatin according to the present disclosure, also apply to the intended uses and methods of the present disclosure.

### Pharmaceutical Compositions

In a further aspect, the present disclosure relates to the administration of an IL-6 signaling inhibitor as described herein in the form of a pharmaceutical composition. Otherwise said, the present disclosure also relates to pharmaceutical compositions comprising an IL-6 signaling inhibitor as described herein.

Typically, an IL-6 signaling inhibitor may be combined with pharmaceutically or physiologically acceptable excipients or carrier, and optionally sustained-release matrices, such as biodegradable polymers, to form therapeutic compositions.

Pharmaceutical compositions provided herein comprise the active agents, i.e., IL-6 signaling inhibitor, in a therapeutically effective amount, i.e., in an amount effective to achieve its intended purpose. As exposed above, the actual effective amount for a particular application will depend, inter alia, on the condition being treated and various other factors well-known in the art such as the age, the weight, the sex of the patient, the presence of other potential aggravating conditions, or the diet. Determination of a therapeutically effective amount of an IL-6 signaling inhibitor of the disclosure is well within the capabilities of those skilled in the art.

The pharmaceutical compositions described herein can be prepared according to techniques known to the skilled person by using an IL-6 signaling inhibitor of the present disclosure in association with a pharmaceutically acceptable excipient or carrier.

These pharmaceutical compositions may comprise one or more pharmaceutically acceptable excipients or carriers. Suitable carriers and excipients and their formulations are described, for example, in Remington: The Science and Practice of Pharmacy, 21^{st} Edition, David B. Troy, ed., Lippicott Williams & Wilkins (2005). By pharmaceutically acceptable carrier is meant a material that is not biologically or otherwise undesirable, i.c., the material is administered to a subject without causing undesirable biological effects or interacting in a deleterious manner with the other components of the pharmaceutical composition in which it is contained.

The pharmaceutical compositions can be in any form deemed appropriate by the skilled person, such as solid, semi-solid, liquid, granular, inhalation or aerosol inhalation.

The liquid forms may be appropriate forms for oral or systemic administration.

Pharmaceutical compositions suitable for oral administration can be capsules, tablets, pills, powders, granules, solutions or suspensions in aqueous or non-aqueous liquids, foam or beaten edible, liquid oil in water emulsions or liquid water in oil emulsions.

For example, for oral administration in capsule or tablet form, the active agents mentioned herein may be combined with pharmaceutically acceptable inert carrier such as ethanol, glycerol, water and similar. There may also be present flavourings, preservative, coloring coating and/or dispersant agents.

Pharmaceutical compositions suitable for parenteral administration may include sterile aqueous or non-aqueous solution for injection which may contain antioxidants, buffers, bacteriostatic and solutes which render the solution isotonic with the blood of the intended recipient, and aqueous or non-aqueous sterile suspensions which may include suspending and thickening agents. These compositions may be sterilized by conventional, well known sterilization techniques.

A parenteral composition may include a solution or suspension of the compounds in a vehicle such as sterile water or a parenterally acceptable oil. Alternatively, the solution can be lyophilized. The lyophilized parenteral pharmaceutical composition can be reconstituted with a suitable solvent just prior to administration.

The pharmaceutical compositions may be presented in single dose or multi-dose containers, for example, sealed ampoules or vials, and may be stored in lyophilized condition requiring only the addition of the sterile liquid carrier, for example water for injections, immediately prior to use. Extemporaneous injection solutions and suspensions may be prepared from powders, granules, lyophilized and sterile compresses.

In the case of parenteral administration, the composition may also be provided with the active ingredients in separate containers that can be suitably admixed according to the desired dosage taking into account the weight, age, gender and health status of the patient in need thereof.

In all cases, the pharmaceutical compositions must be stable under the conditions of manufacture and storage and must be preserved against the contaminating action of microorganisms, such as bacteria and fungi.

All or part of the particular features and embodiments relating to pharmaceutical compositions according to the present disclosure, also apply to the intended uses and methods of the present disclosure.

### Therapeutics uses & Methods of Treatment

In another aspect of the present disclosure, an IL-6 signaling inhibitor or a pharmaceutical composition comprising an IL-6 signaling inhibitor according to the present disclosure may be for use in a method for treating and/or preventing cancer, in particular liposarcoma, in a subject in need thereof, wherein the subject has been previously classified as being affected with a cancer exhibiting recruitment of MDM2 to chromatin.

In some embodiments, the cancer cells exhibiting recruitment of MDM2 to chromatin are further deprived of serine and glycine.

In some embodiments, the present disclosure relates to the use of an IL-6 signaling inhibitor according to the present disclosure for the manufacture of a medicament for the prevention and/or treatment of a cancer, in particular liposarcoma, in a subject in need thereof, wherein the subject has been previously classified as being affected with a cancer exhibiting recruitment of MDM2 to chromatin.

In some other embodiments, the IL-6 signaling inhibitor or the pharmaceutical composition comprising an IL-6 signaling inhibitor according to the present disclosure may be for use in a synergistic prevention and/or treatment of a cancer exhibiting recruitment of MDM2 to chromatin in a subject, wherein the cancer cells exhibiting recruitment of MDM2 to chromatin of the subject are deprived of serine and glycine.

In some other embodiments, the IL-6 signaling inhibitor or the pharmaceutical composition comprising an IL-6 signaling inhibitor according to the present disclosure may be for use in synergistic prevention and/or treatment of a cancer in a subject, wherein the subject has been previously classified as being affected with a cancer exhibiting recruitment of MDM2 to chromatin and wherein the cancer cells exhibiting recruitment of MDM2 to chromatin of the subject are deprived of serine and glycine.

In other embodiments, the IL-6 signaling inhibitor or a pharmaceutical composition comprising an IL-6 signaling inhibitor according to the present disclosure may be for use in a method for treating and/or preventing liposarcoma in a subject in need thereof, wherein the subject has been previously classified as being affected with a liposarcoma exhibiting recruitment of MDM2 to chromatin.

In some embodiments the manufactured medicaments and serine/glycine-deprived diet as described herein may be for a synergistic prevention and/or synergistic treatment of a cancer disease, in particular a cancer exhibiting recruitment of MDM2 to chromatin. As above indicated, “synergy” or “therapeutic synergy” are used when the combination of two conditions at given features or doses is more efficacious than the best of the two conditions alone considering the same features or doses.

Because of the synergistic effect with a serine/glycine-deprived diet, it is expected that the IL-6 signaling inhibitors or pharmaceutical compositions comprising an IL-6 signaling inhibitor of the present disclosure if present in an amount below their usual prescribed effective amount or their therapeutically effective amount as active agent, the effect of the treatment on a subject will be also effective.

In some embodiments, the present disclosure relates to an Interleukin-6 (IL-6) signaling inhibitor, or a pharmaceutical composition comprising such an IL-6 signaling inhibitor, for use in a method for treating and/or preventing a cancer exhibiting recruitment of MDM2 to chromatin in a subject in need thereof, wherein the subject has been previously classified as being affected with a cancer exhibiting recruitment of MDM2 to chromatin, and, optionally, wherein the cancer cells exhibiting recruitment of MDM2 to chromatin of the subject are deprived of serine and glycine.

A further aspect, the present disclosure also relates to methods of treating and/or preventing a cancer in a subject in need thereof, said method comprising at least the steps of:
(a) determining if the subject is affected by a cancer exhibiting a recruitment of MDM2 to chromatin, and
(b) administering to the subject a therapeutically effective amount of an IL-6 signaling inhibitor.

In some embodiments, the methods as described herein also comprise a step of depriving the cancer cells exhibiting recruitment of MDM2 to chromatin of the subject of serine and glycine.

In some embodiments, the methods as described herein also comprise a step of administering a MDM2 inhibitor.

In some embodiments, an IL-6 signaling inhibitor as implemented in the methods as described herein may be selected from an anti-IL-6 inhibitor, an anti-IL-6 receptor inhibitor, an anti-IL-6/IL-6R complex inhibitor, a STAT3 inhibitor or a gp130 inhibitor.

In some embodiments, the present disclosure also relates to a method of treating and/or preventing a cancer in a subject in need thereof, said method comprising at least the steps of:
(a) determining if the subject is affected by a cancer exhibiting a recruitment of MDM2 to chromatin,
(b) optionally depriving the cancer cells of the subject of serine and glycine, and
(c) administering to the subject a therapeutically effective amount of a pharmaceutical composition comprising an IL-6 signaling inhibitor as described herein, thereby treating the cancer.

In some embodiments, the methods of treating and/or preventing a cancer exhibiting recruitment of MDM2 to chromatin in a subject as described herein also comprise a step of administering to the subject a serine/glycine-deprived diet.

In some embodiments, the present disclosure also relates to a method of synergistically preventing and/or treating a cancer exhibiting recruitment of MDM2 to chromatin disease in a subject in need thereof, said method includes administering to the subject a synergistically therapeutic effective amount of at least one IL-6 signaling inhibitor and deprive the cancer cell exhibiting recruitment of MDM2 to chromatin of the subject of serine and glycine, thereby treating the cancer disease in said subject. The methods of the disclosure include observing a prevention or a treatment, such as a relieving, of the cancer disease.

In some embodiments, an IL-6 signaling inhibitor in the method described herein and a serine/glycine deprived diet may be simultaneously, separately or sequentially administered to the subject in need thereof.

In some particular embodiments, an IL-6 signaling inhibitor in the method described herein, a serine/glycine deprived diet and a MDM2 inhibitor may be simultaneously, separately or sequentially administered to the subject in need thereof.

In some other embodiments, the serine/glycine deprived diet is firstly administered and subsequently the IL-6 signaling inhibitor, or the pharmaceutical composition comprising an IL-6 signaling inhibitor as described herein may be administered to the subject.

In particular, the serine/glycine deprived diet may be firstly administered during about 1 day to about 15 days before the administration of the IL-6 signaling inhibitor or the pharmaceutical composition as described herein to the subject in need thereof. Particularly, the serine/glycine-deprived diet may be firstly administered for 1, 2, 3, 4, 5, 6, 7, 8, 9, 10, 11, 12, 13, 14 or 15 days before the administration of the IL-6 signaling inhibitor or the pharmaceutical composition as described herein to the subject in need thereof.

In some other embodiments, the IL-6 signaling inhibitor, or the pharmaceutical composition comprising an IL-6 signaling inhibitor as described herein may be firstly administered to the subject in need thereof and subsequently the serine/glycine deprived diet may be administered. In particular, the IL-6 signaling inhibitor, or the pharmaceutical composition comprising an IL-6 signaling inhibitor as described herein, may be firstly administered during about 1 day to about 15 days before the administration of the IL-6 signaling inhibitor, or the pharmaceutical composition as described herein to the subject in need thereof. Particularly, the IL-6 signaling inhibitor, or the pharmaceutical composition may be firstly administered for 1, 2, 3, 4, 5, 6, 7, 8, 9, 10, 11, 12, 13, 14 or 15 days before the administration of the serine/glycine deprived diet as described herein.

In some other embodiments, the IL-6 signaling inhibitor, or the pharmaceutical composition comprising an IL-6 signaling inhibitor as described herein, and a serine/glycine-deprived diet as disclosed herein may be simultaneously administered to the subject in need thereof.

In particular, the IL-6 signaling inhibitor, or the pharmaceutical composition comprising an IL-6 signaling inhibitor as described herein, and a serine/glycine-deprived diet as disclosed herein may be simultaneously administered for about 1 day to about 15 days to the subject in need thereof, immediately or 1 or 2 days after being determined to be affected by a cancer exhibiting a recruitment of MDM2 to chromatin.

### Screening Methods

The present disclosure also relates to an in vitro method of determining whether a subject is affected with a cancer exhibiting a recruitment of MDM2 to chromatin, wherein said subject is intended for a therapy which comprises an IL-6 signaling inhibitor, comprising:
determining whether MDM2 is localized in the cancer cells nucleus of a biological sample obtained from the subject,
wherein if MDM2 is localized in the cancer cells nucleus of the biological sample it indicates that the subject is affected by a cancer exhibiting recruitment of MDM2 to chromatin.

In the context of the present disclosure, an “alteration” in the level of nuclear-bound MDM2 in a cancer cell means that the level of MDM2 in the nucleus is higher than the level of MDM2 in the nucleus in a control cancer cell. More particularly, an “alteration” in the level of nuclear-bound MDM2 in a cancer cell mean that the level of nuclear-bound MDM2 represent more than about 1% of nuclear-bound MDM2 cells. An “alteration” in the level of nuclear-bound MDM2 in a cancer cell may also simply mean that the localization of MDM2 is at the cell nucleus. In contrast, the localization of MDM2 in a control cancer cell is present in the cytoplasm of said control cancer cell.

In some embodiments, a biological sample may be a whole blood sample, plasma, or serum.

The term “whole blood” refers to blood that has not been centrifuged and therefore contains all of the cells, in particular the red cells, present in the blood.

The following examples are provided for purpose of illustration and not limitation.

### EXAMPLES

### Example 1: Materials and Methods

### Cancer Cell Lines and Culture Conditions

Cancer cell lines culture reagents were purchased from Gibco (Invitrogen). All cancer cell lines were cultured at 37° C. in humidified 5% CO₂ incubator, in DMEM Glutamax (Gibco) medium, supplemented with 10% Fetale Bovine Serum (#8301, Eurobio) and 1% Penicillin-Steeptomycin (10 000 U/mL, Gibco). Except for Jurkat cell lines that were cultured in suspension in RPMI (Gibco) medium.

Cellosaurus XG-6 cell lines (IL6 dependent) were cultured at 37° C. in humidified 5% CO₂ incubator, in RPMI (Gibco) medium, supplemented with 10% Fetale Bovine Serum (#8301, Eurobio) and 1% Penicillin-Steeptomycin (10 000 U/mL, Gibco).

Immortalized Mouse Myoblast cells (C2C12 cell line) were cultured at 37° C. in humidified 5% CO₂ incubator, in DMEM Glutamax (Gibco), supplemented with 10% Fetale Bovine Serum (#8301, Eurobio) and 1% Penicillin-Steeptomycin (10 000 U/mL, Gibco).

After 3 days of culture, the supernatant of the cancer cell lines and mouse myoblast cells was collected and cleared from cell debris by applying centrifugal force at 3000 rpm for 10 min. Supernatant were frozen and stored at −20° C.

The different cancer cell lines exhibiting recruitment of MDM2 to chromatin (C-MDM2 (+)) or not (C-MDM2 (−)) are exposed in Table 1.

TABLE 1
The different cancer cell lines exhibiting,
or not, recruitment of MDM2 to chromatin

| Cancer cell lines | origine | C-MDM2 |
|---|---|---|
| CFPAC | Pancreatic | + (POS) |
| | adenocarcinoma | |
| MDAMB468 | Breast cancer | + (POS) |
| SKMEL5 | Melanoma | + (POS) |
| IB115 | Liposarcoma | + (POS) |
| IB111 | Liposarcoma | + (POS) |
| JURKAT | Leukemia | − (NEG) |
| MCF7 | Breast cancer | − (NEG) |
| ZR751 | Carcinoma | − (NEG) |
| H1299 | Lung Carcinoma | − (NEG) |
| LNCAP | Prostatic | − (NEG) |
| | adenocarcinoma | |
| HPAC | Pancreatic | − (NEG) |
| | adenocarcinoma | |
| MIAPACA | Pancreatic cancer | − (NEG) |

For amino acid starvation experiments, cells were cultured in DMEM lacking serine and glycine (Biological Industries). The media were supplemented with 500 μM Glucose (Sigma-D9434), 550 μM L-Glutamine (Growth factor Kit, ATCC), 300 μM L-Glycine (Sigma-G8898) and 150 μM L-Serine (Sigma S4500) as to obtain a Physiological-like medium and Physiological-like medium without serine and glycine. Prior to their addition to the medium Amino acids and sugar powders were each resuspended in Sterile MilliQ water and filtered (0.45 μm).

Cancer Cell Line Growth Conditions (with DMEM, Liposarcoma Supernatant, DMEM without Serine/Glycine etc.)

50 000 XG-6 cells (IL-6 dependent cell line) were seeded in an equal number in four plastic dishes and cultured in suspension in 2 ml of RPMI (Gibco) medium at 37° C./5% CO2 for 3 days. The fourth dishes' conditions are as follows:
XG-6 cells cultured in 2 ml of RPMI in the presence of 2 ng/ml of recombinant IL-6 (obtained with the method illustrated in Wijdenes et al., Mol Immunol, 28 (1991), p. 1183)
XG-6 cells cultured in 2 ml of RPMI,
XG-6 cells cultured in 2 ml of MCF7 cell lines supernatant as previously obtained,
XG-6 cells cultured in 2 ml of IB115 cell lines supernatant as previously obtained.

### Il-6 Concentration

IL-6 concentration in the supernatant of the different cancer cell lines was measured by an immunoassay (Murine IL-6 Standard TMB ELISA Development Kit Catalog Number: 900-T50) following the manufacturer's instructions.

### Determination of MDM2-Recruitment to Chromatin

### Immunofluorescence Analysis

Cancer cells were fixed with PFA 4% for 15 min then permeabilized for 15 min at RT with PBS Triton 0.1% and blocked with PBS-BSA 0.3% for 1 h at RT before overnight incubation at 4° C. with an anti-mouse monoclonal antibody against MDM2 (MABE340 Millipore). Immunodetection was performed using an Alexa 488-conjugated anti-mouse IgG antibody (Thermo Fisher) for 45 min at room temperature. Cover glasses were mounted with Mowiol (Biovalley) and DAPI (Sigma) before analysis on a Zeiss apotome.

### Chromatin Fractionation Assays

Subcellular fractionation assays were performed essentially as previously described by Riscal et al., 2016. Briefly, cancer cell lines (presented in table 1) were seeded in 150-mm dishes grown in DMEM until they reached 90% confluence. Cells were then washed with PBS, scraped, and lysed in lysis buffer 1 (10 mM Hepes, 10 mM KCl, 1.5 mM MgCl2, 0.34 M sucrose, 10% glycerol, protease inhibitors complete EDTA-free, and 1 mM DTT) and then rapidly centrifuged at 3500 RPM at 4° C. The supernatant containing cytosoluble proteins was stored and then pooled with the fraction containing nucleosoluble proteins (S fraction). Nucleosoluble proteins were recovered by vortexing the pellet upon incubation with lysis buffer 2 (3 mM EDTA (ethylenediaminetetraacetic acid), 0.2 mM EGTA (egtazic acid), and protease inhibitors) for 30 min at 4° C. After 2 washes with buffer 2, chromatin-associated proteins were recovered from the pellet by addition of Laemmli buffer for immunoblotting (C fraction).

### RNA Extraction and RT-qPCR

Total mRNAs were isolated from myoblasts (C2C12 cells) using TRIzol Reagent (Invitrogen) as previously described (Riscal et al. Mol Cell. 2016 Jun. 16; 62 (6): 890-902). Afterwards, Reverse Transcription of 1 μg mRNA into cDNA was performed using SuperScript III Reverse Transcriptase (Invitrogen), according to the manufacturer protocol. Quantification of the produced cDNA was achieve by Real-time quantitative PCRs on a LightCycler 480 SW 1.5 apparatus (Roche) using SYBR Green mix (Ozyme) plus the oligonucleotides of interest, and the following amplification protocol: 45 cycles of 95° C. for 4 s, 60° C. for 10 s, and 72° C. for 15 s. Analysis of the relative gene expression (mRNA copy number) was realized using Ct values and the 2^{-ΔCt} method, normalized on at least two different housekeeping genes (TBP, Tubuline, RPL13a, Gus B, β-microglobuline). Sequence of primers used for PCR are listed in Table 2.

TABLE 2
Primers for qPCR

| | | SEQ ID | | SEQ ID |
|---|---|---|---|---|
| | Forward (5′→3′) | NO. | Reverse (5′→3′) | NO. |
| PSAT | ACTTCCTGTCCAAGCCAGT | 1 | CTGCACCTTGTATTCCAG | 6 |
| | GGA | | GACC | |
| PSPH | GACAGCACGGTCATCAGA | 2 | CGCTCTGTGAGAGCAGCT | 7 |
| | GAAG | | TTGA | |
| PHGDH | CTTACCAGTGCCTTCTCTC | 3 | GCTTAGGCAGTTCCCAGC | 8 |
| | CAC | | ATTC | |
| TBP | ATCAACATCTCAGCAACC | 4 | TTGAAGCTGCGGTACAAT | 9 |
| | CA | | TC | |
| Tubuline | GATCGGTGCTAAGTTCTG | 5 | AGGGACATACTTGCCACC | 10 |
| B5 | GGA | | TGT | |

### IC50 Measurement

Cell survival after SP141, IL-6, IL-6 inhibitor and/or serine/glycine deprived diet treatment (IC50) was determined using the Sulforhodamine B assay (SRB assay). Cells were seeded in 96-well plates (Sarsted) in complete DMEM medium as to obtain triplicates for each conditions (5 000 cells/well). After 24 h, serial dilutions of the indicated compounds were added to the cells. Then 48 h later, cells were fixed by adding a 10% Trichloroacetic acid solution and stained with a 0.4% SRB solution in 1% acetic acid. Fixed SRB was finally dissolved in 10 mM Tris-HCl solution and 560 nm absorbance was read using a PHERAstar FSX plate reader.

### Expression of Target Genes (PHGDH, PSAT 1 and PSPH) and Analyses

### shRNA Assay

Relative quantities of mRNA of MDM2 direct target genes PHGDH, PSAT1 and PSPH were determined by RT qPCR in C2C12 myoblasts cultured alone or in coculture with IB115 cells treated or not for shRNA mediated depletion of human IL-6 (shIL-6). Data were normalized to the corresponding control samples prepared from IB115 cells treated with a control shRNA (mean SD n=3 independent experiments) Statistical significance was evaluated using non parametric Mann Whitney U tests.

### Anti-IL-6 Assay

Relative quantities of mRNA of MDM2 direct target genes PHGDH, PSAT1 and PSPH were determined by RT-qPCR in C2C12 cells culture alone or in coculture with IB115 cells treated or not with a blocking anti-IL-6 antibody (Wijdenes et al., Mol Immunol, 28 (1991), p. 1183),i.e., IL-6 receptor inhibitor such as Bazedoxifène, or STAT3 inhibitors such as C188-9 and Stattic (mean+/−SD; n=3 independent experiments). Statistical significance was evaluated using non-parametric Mann-Whitney U tests.

### Imaging and Counting on Liposarcoma Cells

GFP-IB115 liposarcoma cells (60 000 cells/well) were cultured alone or in coculture with RFP-C2C12 myoblasts (20 000 cells/well) in serine/glycine deprived DMEM medium with 5% serum in 6-well plates. Cell were treated twice a week with 1 μM of anti-IL-6 antibody. The culture lasted 9 days. The three culture assays are as follows:
GFP-IB115 liposarcoma cells cultured in serine/glycine deprived DMEM medium with 5% serum (negative control),
GFP-IB115 liposarcoma cells cocultured with RFP-C2C12 myoblasts in serine/glycine deprived DMEM medium with 5% serum,
GFP-IB115 liposarcoma cells cocultured with RFP-C2C12 myoblasts in serine/glycine deprived DMEM medium with 5% serum+anti-IL-6 antibody.

Cell proliferation was monitored every days using a multi-channel imaging cell cytometer (Celigo®) by acquiring images of each well. The images were then treated using its analysis software to count IB115-GFP cells based on their fluorescence.

Mice Serine Deprivation and Treatment with Anti-IL-6 Antibody

Mice liposarcoma PDX models were established in collaboration with the surgical and pathology departments of the Institut du Cancer de Montpellier (ICM) by inserting a human tumor fragment of approximately 40 mm 3 subcutaneously on 8-week-old CD 1 Foxn 1 nu mice (Charles River).

40 mice were fed with a control diet (called Amino Acid diet; TD 99366 Harlan ENVIGO) or a test diet (Harlan Envigo, TD 130775: diet lacking serine and glycine) during 3 weeks. The diets had equal caloric value (3.9 kCal/g), an equal amount of total amino acids (179.6 g/kg) and are in a form of kibbles for mice. Total food intake was controlled to be identical in all experimental groups. Mice were housed in a pathogen-free barrier facility in accordance with the regional ethics committee for animal warfare (no CEEA-LR-12067). Anti-IL-6 antibody was administered by intra perinoteal i.p. injection at the dose of 100 ug/kg twice a week for 3 weeks. Experiment was done with 10 mice per group, as follows:
10 mice were fed with 5 g/day of the Amino Acid diet (Control),
10 mice were fed with 5 g/day of the test diet (W/O serine),
10 mice were fed with 5 g/day of the Amino Acid diet+injection by i.p. at the dose of 100 μg/kg twice a week,
10 mice were fed with 5 g/day of the test diet+injection by i.p. at the dose of 100 μg/kg twice a week.

Volumetric measurements of xenografted tumors were performed every 3 days by the same person using a manual caliper (volume=length×width²/2). All animals were euthanized when the first animal reached the ethical endpoint (volume =1500 cm3 or ulceration).

### Example 2: Results In Vitro

### Cancer Cells Exhibiting Recruitment of MDM2 to Chromatin Secrete Measurable IL-6

To investigate the importance of MDM2 localization in cancer cells secreting IL-6, it has been analyzed the level of IL-6 secretion in 12 cancer cell lines exhibiting or not a recruitment of MDM2 to chromatin.

Therefore, it was tested 5 cancer cell lines exhibiting a recruitment of MDM2 to chromatin (C-MDM2 (+)) which are CFPAC (Pancreatic adenocarcinoma), MDAMB468 (Breast cancer), SKMEL5 (Melanoma), IB115 (liposarcoma) and IB111 (liposarcoma), and 7 cancer cell lines without exhibiting a recruitment of MDM2 to chromatin (C-MDM2(−)), which are JURKAT (leukemia), MCF7 (Breast cancer), ZR751 (Carcinoma), H1299 (Lung Carcinoma), LNCAP (Prostatic adenocarcinoma), HPAC (Pancreatic adenocarcinoma) and MIAPACA (Pancreatic cancer) (see Table 1).

These 12 cancer cell lines all overexpress MDM2 (https://sites.broadinstitute.org/ccle).

As shown in FIG. 1A, CFPAC, MDAMB468, SKMEL5, IB 115 and IB111 secrete a high level of IL-6, of about 1600 pg/ml, 4650 pg/ml, 1300 pg/ml,5000 pg/ml and 7000 nM of IL-6, respectively. In contrast, JURKAT, MCF7, ZR751, H1299, LNCAP, HPAC and MIAPACA cells secrete a very low level of IL-6 or do not secrete IL-6. Further, as shown in FIG. 1B and FIG. 9, IL-6 secretion is highly dependent on the localization of MDM2 in the nucleus, independently of MDM2 expression level.

Thus, cancer cell lines exhibiting recruitment of MDM2 to chromatin, like liposarcoma, secrete a high level of IL-6 whereas cancer cell lines without MDM2 localized in the nucleus, do not secrete IL-6 or at least a non-significative low level.

Subsequently, it was evaluated whether the secretion of IL-6 by cancer cell lines is effectively link with the subcellular localization of MDM2.

As shown in FIG. 2A, it was observed that all cancer cell lines secreting a high level of IL-6, ranging from 2 to 8, have also an overexpression of MDM2, ranging from 1 to 6. In contrast, in FIG. 2B, it is observed that all cancer cell lines overexpressing MDM2, ranging from 4 to 8, do not necessarily express IL-6, ranging from-13 to 6.

Therefore, cancer cells expressing IL-6 have an overexpression of MDM2 whereas cancer cells overexpressing MDM2 do not necessarily express IL-6.

These results indicate that the sole overexpression of MDM2 in cancer cell is not correlate with the high secretion of IL-6. Thus, it is confirmed that the localization of MDM2 to chromatin is important and activate a cellular pathway allowing the secretion of IL-6.

Further, it has been observed that cancer cell lines which exhibits a recruitment of MDM2 to chromatin do not necessarily have an overexpression of MDM2 in the cell (shown on FIG. 10).

### IL-6 Promotes the IL-6-Dependent Cancer Cells Growth

Then, to investigate the role of IL-6 in the growth of cancer, it was tested the growth of an IL-6 dependent cell line (XG-6) in presence or absence of IL-6 recombinant (positive and negative control, respectively) and in presence of MCFY7 medium or IB115 medium. As shown in FIG. 3, XG-6 cells (IL-6 dependent cell line) grow in the presence of recombinant IL-6 (+IL6) to more than 150000 AU, whereas no growth is observed without recombinant IL-6 (-IL6).

It was further observed that in the presence of MCF7 medium supernatant, XG-6 cells grow to about 100000 AU, and in the presence of IB115 medium supernatant, XG-6 cells grow to more than 150000 AU.

Therefore, these data support the fact that the growth of IL-6-dependent cells is promoted by IL-6 and more specifically by IL-6 secreted by cancer cells exhibiting recruitment of MDM2 to chromatin (C-MDM2 (+)), such as liposarcoma cell lines (IB115).

### Human IL-6 is Involved in the Serine Synthesis by Myoblast Cells

As shown in FIGS. 4, 5 and 11, relative mRNA level of PHGDH in myocytes alone is about 1 AU whereas relative mRNA level for myocytes cultured with IB115 liposarcoma cell supernatant (LPS) is significantly higher than myocytes alone assay (about 4.4 AU—FIG. 4 or about 4 AU—FIG. 5). In contrast, relative mRNA level of PHGDH in myocytes cultured with LPS and treated with shIL-6 (about 1.75 AU—FIG. 4) or anti-IL-6 antibody assay (about 2.5 AU—FIG. 5) is significantly lower than the myocyte cultured with LPS assay. Similarly, using IL-6R inhibitor (BZA) on the co-culture myocytes with LPS (about 0.6 A.U—FIG. 11) or STAT3 inhibitor (about 0.5 A.U for C188-9—FIG. 11) reduce PHGDH relative mRNA level compared to the myocytes cultured with LPS without inhibitors (1.6 A.U—FIG. 11).

Regarding PSAT1 gene, relative mRNA level of PSAT in myocytes alone is about 1 AU whereas relative mRNA level for myocytes cultured with LPS is significantly higher than the myocytes alone assay (about 1.75 AU—FIG. 4 or about 3.25 AU—FIG. 5). In contrast, relative mRNA level of PSAT in myocytes cultured with LPS and treated with shIL-6 assay (about 1 AU—FIG. 4) or anti-IL-6 antibody assay (about 2.5 AU—FIG. 5) is significantly lower than the myocyte cultured with LPS. Similarly, using the IL-6R inhibitor (BZA) on the co-culture myocytes with LPS (about 1.3 A.U—FIG. 11) or STAT3 inhibitors (about 1.3 for C188-9 or 1.5 A.U for Stattic—FIG. 11) reduce PSAT1 relative mRNA level compared to the myocytes cultured with LPS without inhibitors (about 2 A.U—FIG. 11).

Regarding PSPH gene, relative mRNA level of PSPH in myocytes alone is about 1 AU whereas relative mRNA level for myocytes cultured with LPS is significantly higher than the myocytes alone assay (about 2.5 AU—FIG. 4 or about 3 AU—FIG. 5). In contrast, relative mRNA level of PSPH in myocytes cultured with LPS and treated with shIL-6 (about 1 AU) or anti-IL-6 antibody (about 1.75 AU—FIG. 5) is significantly lower than the myocyte cultured with LPS without inhibitors. Similarly, using IL-6R inhibitor (BZA) on the co-culture myoblasts with LPS (about 1.8 A.U—FIG. 11) or STAT3 inhibitors (about 1.5 for C188-9 or 1.75 A.U for Stattic—FIG. 11) reduce PSPH relative mRNA level compared to the myocytes cultured with LPS without inhibitors (about 3.1 A.U).

Therefore, these data support the notion that in mouse myoblasts (C2C12 cells) cocultured with human liposarcoma cells, gene transcription of the key enzymes PHGDH, PSAT et PSPH for serine synthesis is enhanced by human IL-6. Hence, myoblast cells secrete serine in presence of IL-6 secreted by cancer cells exhibiting recruitment of MDM2 to chromatin, such as liposarcoma cells.

IL-6-Stimulated Myoblast Cells Provide Liposarcoma Cells with Serine and Sustain their Growth

It can be observed on FIG. 6 that the growth of LPS cells cultured in a serine/glycine-deprived medium is decreased from 100% of living cells to 40% after 9 days. The growth of LPS cells cocultured with myoblasts in a serine/glycine-deprived medium is increased from 40% of living cells to 75% after 9 days. Further, the growth of LPS cells cocultured with myoblasts in the presence of anti-IL-6 antibody in a serine/glycine-deprived medium is decreased from 75% of living cells to 40% after 9 days.

Therefore, liposarcoma cells in absence of serine do sustain their growth whereas in the same condition and cocultured with serine-secreted myoblasts, liposarcoma cells sustain their growth. Otherwise said, serine, in particular serine secreted by myoblasts, allows liposarcoma cells to growth even without external serine/glycine in the culture medium. Indeed, it must be noted that the addition of anti-IL-6 antibodies in the culture medium does not allow the liposarcoma cells to sustain.

### The MDM2 Inhibitor, SP141, Inhibits the Growth of Cancer Cells Exhibiting Recruitment of MDM2 to Chromatin

To further delineate the function of MDM2 in cancer cells exhibiting recruitment of MDM2 to chromatin, it was examined cell viability of several cancer cell lines with (C-MDM2 (+)) or without chromatin-bound MDM2 (C-MDM2 (−)) upon treatment with a MDM2 inhibitor, i.e., SP141. As shown in FIG. 8, it was observed an important loss of cell viability of cancer cells C-MDM2 (+) as soon as 24 hours after treatment with low concentrations of SP141.

### Example 3: Results In Vivo

### Serinelglycine Deprivation and Treatment with anti-IL-6 Antibody have a Synergistic Anti-Tumor Effect on a Human Liposarcoma

It can be observed on FIG. 7 that tumor size on mice at day 24 is drastically reduced between the control condition (1200 mm³) and the anti-IL-6 antibody (aIL-6) assay (600 mm³). Further, it can be observed that the tumor size is also decreased under for the without W/O serine condition (640 mm³) and further decreased under the combined anti-IL-6 antibody (aIL-6) AND W/O serine treatment (260 mm³) compared to the control assay.

Therefore, these data support the fact that a treatment with an anti-IL-6 antibody as stand-alone is effective on growth inhibition of a PDX human tumor expressing MDM2 at chromatin and that such a specific anti-IL-6 treatment combines favorably with a serine/glycine-deprived diet.

### Example 4: Conclusions

Although it was already known that chromatin-bound MDM2 was a key regulator of serine synthesis in cancer cells (Riscal et al., 2016), the molecular mechanisms that control the expression of genes involved in serine metabolisms remain unknown. Thus, providing an effective treatment against cancer exhibiting recruitment of MDM2 to chromatin remains difficult.

As shown in the examples, the inventors demonstrate that there is no link between the total amount of MDM2 in the cell (and/or its gene amplification) and its localization or not to chromatin. However, it is exclusively when MDM2 is localized to chromatin (C-MDM2 (+)) that cell metabolism is drastically modified, with an increased need for serine. Accordingly, the recruitment of MDM2 to chromatin is a key factor in adaptive tumor metabolism.

Among the metabolic changes controlled by C-MDM2 is the intra-tumoral stimulation of IL-6 synthesis, which IL-6 will remotely trigger the overproduction of serine in myocytes. Therefore, the inhibition of the production of IL-6, by the cancer cells exhibiting recruitment of MDM2 to chromatin, through inhibitors of the IL-6 signalization causes a down regulation of the serine pathway in myoblasts and thus its production (FIGS. 4, 5 and 11).

Thus, the present disclosure provides a method for treating and/or preventing cancer exhibiting recruitment of MDM2 to chromatin, in particular liposarcoma, in a subject which has been classified as affected by a cancer exhibiting recruitment of MDM2 to chromatin and treated with an Interleukin-6 (IL-6) signaling inhibitor. The present disclosure further demonstrates that treatment with an IL-6 signaling inhibitor in combination with a treatment wherein the cancer cells are deprived of any source of serine and any source of glycine, increase the efficacy of the treatment (FIG. 7).

In particular, the present disclosure shown surprisingly that myoblast cells in presence of IL-6 synthesis, and secret, serine. Based on this discovery, the inventors have tested different conditions to confirm this finding and surprisingly found that the combination of such a serine/glycine deprivation and the injection of anti-IL-6 antibody allows a better reduction of the human tumor size and thus to treat a cancer exhibiting recruitment of MDM2 to chromatin.

### REFERENCES

Biderman L, Manley JL, Prives C. Mdm2 and MdmX as Regulators of Gene Expression. Genes Cancer. 2012; 3(3-4):264-273. doi:10.1177/1947601912455331
Boulanger MJ, Chow DC, Brevnova EE, Garcia KC. Hexameric structure and assembly of the interleukin-6/IL-6 alpha-receptor/gp130 complex. Science. 2003 Jun. 27; 300(5628):2101-4. doi:10.1126/science.1083901. Erratum in: Science. 2003 Aug. 15; 301(5635):918. PMID: 12829785.
Boerner P, Lafond R, Lu WZ, Brams P, Royston I. Production of antigen-specific human monoclonal antibodies from in vitro-primed human splenocytes. J Immunol. 1991 Jul. 1; 147(1):86-95. PMID: 2051030.
Cissé MY, Pyrdziak S, Firmin N, Gayte L, Heuillet M, Bellvert F, Fuentes M, Delpech H, Riscal R, Arena G, Chibon F, Le Gellec S, Maran-Gonzalez A, Chateau MC, Theillet C, Carrere S, Portais JC, Le Cam L, Linares LK. Targeting MDM2-dependent serine metabolism as a therapeutic strategy for liposarcoma. Sci Transl Med. 2020 Jun. 10; 12(547):eaay2163. doi: 10.1126/scitranslmed.aay2163. PMID: 32522803.
EP 592,106
EP 519,596
EP 125,023
Fishwild, D., O'Donnell, S., Bengoechea, T. et al. High-avidity human IgGκ monoclonal antibodies from a novel strain of minilocus transgenic mice. Nat Biotechnol 14, 845-851 (1996). https://doi.org/10.1038/nbt0796-845
Genovese MC, van der Heijde D, Lin Y, St John G, Wang S, van Hoogstraten H, Gómez-Reino JJ, Kivitz A, Maldonado-Cocco JA, Seriolo B, Stanislav M, Burmester GR. Long-term safety and efficacy of sarilumab plus methotrexate on disease activity, physical function and radiographic progression: 5 years of sarilumab plus methotrexate treatment. RMD Open. 2019 Aug. 1; 5(2):e000887. doi: 10.1136/rmdopen-2018-000887. PMID: 31452928; PMCID: PMC6691511.
Gilbert LA, Larson MH, Morsut L, Liu Z, Brar GA, Torres SE, Stern-Ginossar N, Brandman O, Whitehead EH, Doudna JA, Lim WA, Weissman JS, Qi LS. CRISPR-mediated modular RNA-guided regulation of transcription in eukaryotes. Cell. 2013 Jul. 18; 154(2):442-51. doi: 10.1016/j.cell.2013.06.044. Epub 2013 Jul. 11. PMID: 23849981; PMCID: PMC3770145.
Hamdi A, Colas P. Yeast two-hybrid methods and their applications in drug discovery. Trends Pharmacol Sci. 2012 February; 33(2):109-18. doi: 10.1016/j.tips.2011.10.008. Epub 2011 Nov. 28. PMID: 22130009.
Heinis, C., Rutherford, T., Freund, S. et al. Phage-encoded combinatorial chemical libraries based on bicyclic peptides. Nat Chem Biol 5, 502-507 (2009). https://doi.org/10.1038/nchembio.184
Hennie R. Hoogenboom, Greg Winter. By-passing immunisation: Human antibodies from synthetic repertoires of germline VH gene segments rearranged in vitro. Journal of Molecular Biology, Volume 227, Issue 2, 1992, Pages 381-388, ISSN 0022-2836, https://doi.org/10.1016/0022-2836(92)90894-P.
Jayasena SD. Aptamers: an emerging class of molecules that rival antibodies in diagnostics. Clin Chem. 1999 September; 45(9):1628-50. PMID: 10471678.
Jones, P., Dear, P., Foote, J. et al. Replacing the complementarity-determining regions in a human antibody with those from a mouse. Nature 321, 522-525 (1986). https://doi.org/10.1038/321522a0
Locasale JW. Serine, glycine and one-carbon units: cancer metabolism in full circle. Nat Rev Cancer. 2013 August; 13(8):572-83. doi: 10.1038/nrc3557. Epub 2013 Jul. 4. PMID: 23822983; PMCID: PMC3806315.
Lonberg, N., Taylor, L., Harding, F. et al. Antigen-specific human antibodies from mice comprising four distinct genetic modifications. Nature 368, 856-859 (1994). https://doi.org/10.1038/368856a0
Marks JD, Hoogenboom HR, Bonnert TP, McCafferty J, Griffiths AD, Winter G. By-passing immunization. Human antibodies from V-gene libraries displayed on phage. J Mol Biol. 1991 Dec. 5; 222(3):581-97. doi: 10.1016/0022-2836 (91) 90498-u. PMID: 1748994.
Marks, J., Griffiths, A., Malmqvist, M. et al. By-Passing Immunization: Building High Affinity Human Antibodies by Chain Shuffling. Nat Biotechnol 10, 779-783 (1992). https://doi.org/10.1038/nbt0792-779
Mease, P.J., Gottlieb, A.B., Berman, A., Drescher, E., Xing, J., Wong, R. and Banerjee, S. (2016), The Efficacy and Safety of Clazakizumab, an Anti-Interleukin-6 Monoclonal Antibody, in a Phase IIb Study of Adults With Active Psoriatic Arthritis. Arthritis & Rheumatology, 68:2163-2173. https://doi.org/10.1002/art.39700
Neuberger, M., Williams, G., Mitchell, E. et al. A hapten-specific chimaeric IgE antibody with human physiological effector function. Nature 314, 268-270 (1985). https://doi.org/10.1038/314268a0
Neuberger, M. Generating high-avidity human Mabs in mice. Nat Biotechnol 14, 826 (1996). https://doi.org/10.1038/nbt0796-826a
Nishimoto N, Miyasaka N, Yamamoto K, Kawai S, Takeuchi T, Azuma J. Long-term safety and efficacy of tocilizumab, an anti-IL-6 receptor monoclonal antibody, in monotherapy, in patients with rheumatoid arthritis (the STREAM study): evidence of safety and efficacy in a 5-year extension study. Ann Rheum Dis. 2009 October; 68(10):1580-4. doi: 10.1136/ard.2008.092866. Epub 2008 Nov. 19. PMID: 19019888; PMCID: PMC2732899.
Platt RJ, Chen S, Zhou Y, Yim MJ, Swiech L, Kempton HR, Dahlman JE, Parnas O, Eisenhaure TM, Jovanovic M, Graham DB, Jhunjhunwala S, Heidenreich M, Xavier RJ, Langer R, Anderson DG, Hacohen N, Regev A, Feng G, Sharp PA, Zhang F. CRISPR-Cas9 knockin mice for genome editing and cancer modeling. Cell. 2014 Oct. 9; 159(2):440-55. doi: 10.1016/j.cell.2014.09.014. Epub 2014 Sep. 25. PMID: 25263330; PMCID: PMC4265475.
Presta LG. Antibody engineering. Curr Opin Biotechnol. 1992 August; 3(4):394-8. doi: 10.1016/0958-1669 (92) 90168-i. PMID: 1368441.
Riechmann L, Clark M, Waldmann H, Winter G. Reshaping human antibodies for therapy. Nature. 1988 Mar. 24; 332(6162):323-7. doi: 10.1038/332323a0. PMID: 3127726.
Riscal R, Schrepfer E, Arena G, Cissé MY, Bellvert F, Heuillet M, Rambow F, Bonneil E, Sabourdy F, Vincent C, Ait-Arsa I, Levade T, Thibaut P, Marine JC, Portais JC, Sarry JE, Le Cam L, Linares LK. Chromatin-Bound MDM2 Regulates Serine Metabolism and Redox Homeostasis Independently of p53. Mol Cell. 2016 Jun. 16; 62(6):890-902
Rossi JF, Négrier S, James ND, Kocak I, Hawkins R, Davis H, Prabhakar U, Qin X, Mulders P, Berns B. A phase I/II study of siltuximab (CNTO 328), an anti-interleukin-6 monoclonal antibody, in metastatic renal cell cancer. Br J Cancer. 2010 Oct. 12; 103(8):1154-62. doi: 10.1038/sj.bjc.6605872. Epub 2010 Aug. 31. PMID: 20808314; PMCID: PMC2967052.
SANBROOK et al, “Molecular Cloning: A Laboratory Manual,” Second Edition, Cold Spring Harbor Laboratory Press, 1989
Sheets MD, Amersdorfer P, Finnern R, Sargent P, Lindquist E, Schier R, Hemingsen G, Wong C, Gerhart JC, Marks JD. Efficient construction of a large nonimmune phage antibody library: the production of high-affinity human single-chain antibodies to protein antigens. Proc Natl Acad Sci U S A. 1998 May 26; 95(11):6157-62. doi: 10.1073/pnas.95.11.6157. Erratum in: Proc Natl Acad Sci U S A 1999 Jan. 19; 96(2):795. Lindqvist E [corrected to Lindquist E]. PMID: 9600934; PMCID: PMC27609.
Tuerk C, Gold L. Systematic evolution of ligands by exponential enrichment: RNA ligands to bacteriophage T4 DNA polymerase. Science. 1990 Aug. 3; 249(4968):505-10. doi: 10.1126/science.2200121. PMID: 2200121.
U.S. Pat. No. 5,750,373
U.S. Pat. Nos. 5,225,539
U.S. Pat. Nos 5,530,101
U.S. Pat. Nos 5,585,089
U.S. Pat. No. 5,565,332
U.S. Pat. Nos. 6,573,099
U.S. Pat. Nos. 6,506,559
Vaughan, T., Williams, A., Pritchard, K. et al. Human Antibodies with Sub-nanomolar Affinities Isolated from a Large Non-immunized Phage Display Library. Nat Biotechnol 14, 309-314 (1996). https://doi.org/10.1038/nbt0396-309.
Wade M, Li YC, Wahl GM. MDM2, MDMX and p53 in oncogenesis and cancer therapy. Nat Rev Cancer. 2013 February; 13(2):83-96. doi: 10.1038/nrc3430. Epub 2013 Jan. 10. PMID: 23303139; PMCID: PMC4161369.
Wijdenes J, Clement C, Klein B, Morel-Fourrier B, Vita N, Ferrara P, Peters A. Human recombinant dimeric IL-6 binds to its receptor as detected by anti-IL-6 monoclonal antibodies. Mol Immunol. 1991 November; 28(11):1183-92. doi: 10.1016/0161-5890(91)90004-4. PMID: 1720501.
WO1991009967A1
WO 96/02576
WO 01/36646
WO 99/32619
WO 01/68836
Wu X, Cao Y, Xiao H, Li C, Lin J. Bazedoxifene as a Novel GP130 Inhibitor for Pancreatic Cancer Therapy. Mol Cancer Ther. 2016 November; 15(11):2609-2619. doi: 10.1158/1535-7163.MCT-15-0921. Epub 2016 Aug. 17. PMID: 27535971; PMCID: PMC5310670.
Xu, Z., Bouman-Thio, E., Comisar, C., Frederick, B., Van Hartingsveldt, B., Marini, J.C., Davis, H.M. and Zhou, H. (2011), Pharmacokinetics, pharmacodynamics and safety of a human anti-IL-6 monoclonal antibody (sirukumab) in healthy subjects in a first-in-human study. British Journal of Clinical Pharmacology, 72:270-281. https://doi.org/10.1111/j.1365-2125.2011.03964.x
Zhuang, Shougang (2013). “Regulation of STAT signaling by acetylation”. Cellular Signalling. 25(9):1924-1931. doi: 10.1016/j.cellsig. 2013.05.007. PMC 4550442. PMID 23707527

1. A method of treating and/or preventing cancer in a subject in need thereof wherein the subject has been previously classified as being affected with a cancer exhibiting recruitment of MDM2 to chromatin, said method comprising at least a step of administering to the subject a Interleukin-6 (IL-6) signaling inhibitor selected from an IL-6 inhibitor, an IL-6 receptor inhibitor, an IL-6/IL-6 receptor complex inhibitor, a gp130 inhibitor and a STAT3 inhibitor.

2. The method according to claim 1, wherein the cancer cells are further deprived of serine and glycine.

3. The method according to claim 1, wherein the cancer exhibiting recruitment of MDM2 to chromatin is selected from the group comprising; bone cancer, brain cancer, ovary cancer, breast cancer, lung cancer, colorectal cancer, osteosarcoma, skin cancer, malignant hemopathies, pancreatic cancer, prostate cancer and liposarcoma, in particular the cancer exhibiting recruitment of MDM2 to chromatin is liposarcoma.

4. The method according to claim 1, the IL-6 inhibitor being an anti-IL-6 antibody, or an anti-sense oligonucleotide directed to IL-6.

5. The method according to claim 1, the IL-6 receptor inhibitor being an anti-IL-6 receptor antibody, or an anti-sense oligonucleotide directed to IL-6 receptor.

6. The method according to claim 1, the IL-6/IL-6 receptor complex inhibitor being an anti-IL-6/IL-6 receptor complex antibody, or an anti-sense oligonucleotide directed to IL-6/IL-6 receptor complex.

7. The method according to claim 1, wherein the gp130 inhibitor is selected from an anti-gp130 antibody, bazedoxifene and an anti-sense oligonucleotide directed to gp130.

8. The method according to claim 4, wherein the anti-IL-6 antibody is a monoclonal anti-IL-6 antibody.

9. The method according to claim 5, wherein the anti-IL-6 receptor antibody is selected from tocilizumab, sarilumab and TZLS-501.

10. The method according to claim 6, wherein the anti-IL-6/IL-6 receptor complex antibody is TZLS-501.

11. The method according to claim 1, wherein the subject is further treated with a MDM2 inhibitor.

12. A method for treating and/or preventing cancer in a subject in need thereof, wherein the subject has been previously classified as being affected with a cancer exhibiting recruitment of MDM2 to chromatin, the method comprising at least a step of administering to the subject a pharmaceutical composition comprising (i) an IL-6 signaling inhibitor selected from an IL-6 inhibitor, an IL-6 receptor inhibitor, an IL-6/IL-6 receptor complex, a gp130 inhibitor and a STAT3 inhibitor, and (ii) a pharmaceutically acceptable carrier.

13. The method according to claim 12, wherein the cancer cells are further deprived of serine and glycine.

14. The method according to claim 1, wherein the subject is a human.

15. An in vitro method of determining whether a subject is affected with a cancer exhibiting a recruitment of MDM2 to chromatin, wherein said subject is intended for a therapy which comprises an IL-6 signaling inhibitor, comprising:
determining whether MDM2 is localized in the cancer cells nucleus of a biological sample obtained from the subject,
wherein if MDM2 is localized in the cancer cell nucleus of the biological sample, it indicates that the subject is affected by a cancer exhibiting recruitment of MDM2 to chromatin.

16. The IL-6 signaling inhibitor for its use according to claim 3, the cancer exhibiting recruitment of MDM2 to chromatin being liposarcoma.

17. The IL-6 signaling inhibitor for its use according to claim 8, wherein the anti-IL-6 antibody is selected from sirukumab, siltuximab, olokizumab, or clazakizumab.

18. The method according to claim 12, wherein the subject is a human.

19. A method of treating and/or preventing a cancer in a subject in need thereof, said method comprising at least the steps of:
(a) determining if the subject is affected by a cancer exhibiting a recruitment of MDM2 to chromatin, and
(d) administering to the subject a therapeutically effective amount of a pharmaceutical composition comprising an IL-6 signaling inhibitor selected from an IL-6 inhibitor, an IL-6 receptor inhibitor, an IL-6/IL-6 receptor complex inhibitor, a gp130 inhibitor and a STAT3 inhibitor.

20. The method according to claim 19, wherein the method further comprises a step of depriving the cancer cells of the subject of serine and glycine before step (d).


SEQUENCE LISTING (11 sequences)

SEQ ID NO: 1 - DNA, 22 bp, synthetic construct
  source 1..22: mol_type = other DNA
acttcctgtccaagccagtgga

SEQ ID NO: 2 - DNA, 22 bp, synthetic construct
  source 1..22: mol_type = other DNA
gacagcacggtcatcagagaag

SEQ ID NO: 3 - DNA, 22 bp, synthetic construct
  source 1..22: mol_type = other DNA
cttaccagtgccttctctccac

SEQ ID NO: 4 - DNA, 20 bp, synthetic construct
  source 1..20: mol_type = other DNA
atcaacatctcagcaaccca

SEQ ID NO: 5 - DNA, 21 bp, synthetic construct
  source 1..21: mol_type = other DNA
gatcggtgctaagttctggga

SEQ ID NO: 6 - DNA, 22 bp, synthetic construct
  source 1..22: mol_type = other DNA
ctgcaccttgtattccaggacc

SEQ ID NO: 7 - DNA, 22 bp, synthetic construct
  source 1..22: mol_type = other DNA
cgctctgtgagagcagctttga

SEQ ID NO: 8 - DNA, 22 bp, synthetic construct
  source 1..22: mol_type = other DNA
gcttaggcagttcccagcattc

SEQ ID NO: 9 - DNA, 20 bp, synthetic construct
  source 1..20: mol_type = other DNA
ttgaagctgcggtacaattc

SEQ ID NO: 10 - DNA, 21 bp, synthetic construct
  source 1..21: mol_type = other DNA
agggacatacttgccacctgt

SEQ ID NO: 11 - DNA, 23 bp, synthetic construct
  source 1..23: mol_type = other DNA
gacactattttaattatttttaa